SEQUENCE LISTING (72 sequences)

SEQ ID NO: 1 - protein, 107 aa, Artificial Sequence
  TPP-1361 CD47 VL
NIQMTQSPSSLSASVGDRVTITCQASQDIHRYLSWFQQDPGTVPQHLIYRESRFVDGVPSRFSGSGSGTEFTLTISSLQPEDFATYYCLQYDEFPYTFGGGTKVEIK

SEQ ID NO: 2 - protein, 120 aa, Artificial Sequence
  TPP-1361 CD47 VH
QMQLVQSGAEVKKPGSSVKVSCKASGFNIKDYYLHWVRQAPGQGLEWMGWIDPDQGDTYYAQKFQGRVTITRDRSTSTAYMELASLTAEDTAVYYCNAAYGESSYPMDYWGQGTLVTVSS

SEQ ID NO: 3 - protein, 107 aa, Artificial Sequence
  TPP-1360 CD47 VL
NIQMTQSPSSLSASVGDRVTITCRASQDIHRYLSWFQQKPGKVPKHLIYRESRFVDGVPSRFSGSGSGTEFTLTISSLQPEDFATYYCLQYDEFPYTFGGGTKVEIK

SEQ ID NO: 4 - protein, 120 aa, Artificial Sequence
  TPP-1360 CD47 VH
QMQLVQSGAEVKKPGSSVKVSCKASGFNIKDYYLHWVRQAPGKGLEWMGWIDPDQGDTYYAQKFQGRVTITRDRSTSTAYMELRSLRAEDTAVYYCNAAYGESSYPMDYWGQGTLVTVSS

SEQ ID NO: 5 - protein, 107 aa, Artificial Sequence
  TPP-1367 CD47 VL
NIQMTQSPSSLSASVGDRVTITCRASQDIHRYLSWFQQKPGKVPKHLIYRANRLVSGVPSRFSGSGSGTEFTLTISSLQPEDFATYYCLQYDEFPYTFGGGTKVEIK

SEQ ID NO: 6 - protein, 120 aa, Artificial Sequence; identical to SEQ ID NO: 4
  TPP-1367 CD47 VH

SEQ ID NO: 7 - protein, 106 aa, Artificial Sequence [sequence omitted]

SEQ ID NO: 8 - protein, 121 aa, Artificial Sequence [sequence omitted]

SEQ ID NO: 9 - protein, 107 aa, Artificial Sequence
  90002 VL
NIQMTQSPSAMSASVGDRVTITCKASQDIHRYLSWFQQKPGKVPKHLIYRANRLVSGVPSRFSGSGSGTEFTLTISSLQPEDFATYYCLQYDEFPYTFGGGTKVEIK

SEQ ID NO: 10 - protein, 120 aa, Artificial Sequence
  90002 VH
QMQLVQSGAEVKKTGSSVKVSCKASGFNIKDYYLHWVRQAPGQALEWMGWIDPDQGDTEYAQKFQDRVTITRDRSMSTAYMELSSLRSEDTAMYYCNAAYGSSSYPMDYWGQGTTVTVSS

SEQ ID NO: 11 - protein, 214 aa, Artificial Sequence
  90002 WHOLE LC / IgG1
NIQMTQSPSAMSASVGDRVTITCKASQDIHRYLSWFQQKPGKVPKHLIYRANRLVSGVPSRFSGSGSGTEFTLTISSLQPEDFATYYCLQYDEFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 450 aa, Artificial Sequence
  90002 WHOLE HC / IgG1
QMQLVQSGAEVKKTGSSVKVSCKASGFNIKDYYLHWVRQAPGQALEWMGWIDPDQGDTEYAQKFQDRVTITRDRSMSTAYMELSSLRSEDTAMYYCNAAYGSSSYPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 213 aa, Artificial Sequence
  LC - WHOLE RITUXIMAB
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 451 aa, Artificial Sequence
  HC - WHOLE RITUXIMAB
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 213 aa, Artificial Sequence
  CD20 WHOLE LC
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVAIFPPSDERLKSGTASVVCVLNNFYPREAKVQWKVDNALQSGNSQESVTEQDSKDSTYSLSSRLTLSKADYEKHKVYACEVTHQGLSSPVTKSFNRGEC

SEQ ID NO: 16 - protein, 451 aa, Artificial Sequence
  CD20 WHOLE HC
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAWLGCEVTDYFPEPVTVSWNSGALTSGVHTFPAVLESSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 17 - protein, 214 aa, Artificial Sequence
  TPP-1361 CD47 WHOLE LC
NIQMTQSPSSLSASVGDRVTITCQASQDIHRYLSWFQQDPGTVPQHLIYRESRFVDGVPSRFSGSGSGTEFTLTISSLQPEDFATYYCLQYDEFPYTFGGGTKVEIKRTVAAPSVFIFPPSDEELKSGTASVVCWLNNFYPREAKVQWKVDNALQSGNSEESVTEQDSKDSTYSLSSTLELSKADYEKHKVYACEVTHQGLSSPVTKSFNRGEC

SEQ ID NO: 18 - protein, 450 aa, Artificial Sequence
  TPP-1361 CD47 WHOLE HC
QMQLVQSGAEVKKPGSSVKVSCKASGFNIKDYYLHWVRQAPGQGLEWMGWIDPDQGDTYYAQKFQGRVTITRDRSTSTAYMELASLTAEDTAVYYCNAAYGESSYPMDYWGQGTLVTVSSASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 19 - protein, 214 aa, Artificial Sequence
  TPP-1360 CD47 WHOLE LC
NIQMTQSPSSLSASVGDRVTITCRASQDIHRYLSWFQQKPGKVPKHLIYRESRFVDGVPSRFSGSGSGTEFTLTISSLQPEDFATYYCLQYDEFPYTFGGGTKVEIKRTVAAPSVFIFPPSDEELKSGTASVVCWLNNFYPREAKVQWKVDNALQSGNSEESVTEQDSKDSTYSLSSTLELSKADYEKHKVYACEVTHQGLSSPVTKSFNRGEC

SEQ ID NO: 20 - protein, 450 aa, Artificial Sequence
  TPP-1360 CD47 WHOLE HC
QMQLVQSGAEVKKPGSSVKVSCKASGFNIKDYYLHWVRQAPGKGLEWMGWIDPDQGDTYYAQKFQGRVTITRDRSTSTAYMELRSLRAEDTAVYYCNAAYGESSYPMDYWGQGTLVTVSSASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 21 - protein, 214 aa, Artificial Sequence
  TPP-1367 CD47 WHOLE LC
NIQMTQSPSSLSASVGDRVTITCRASQDIHRYLSWFQQKPGKVPKHLIYRANRLVSGVPSRFSGSGSGTEFTLTISSLQPEDFATYYCLQYDEFPYTFGGGTKVEIKRTVAAPSVFIFPPSDEELKSGTASVVCWLNNFYPREAKVQWKVDNALQSGNSEESVTEQDSKDSTYSLSSTLELSKADYEKHKVYACEVTHQGLSSPVTKSFNRGEC

SEQ ID NO: 22 - protein, 450 aa, Artificial Sequence; identical to SEQ ID NO: 20
  TPP-1367 CD47 WHOLE HC

SEQ ID NO: 23 - protein, 107 aa, Artificial Sequence
  anti-CD47 IgG1 LC Constant Region
RTVAAPSVFIFPPSDEELKSGTASVVCWLNNFYPREAKVQWKVDNALQSGNSEESVTEQDSKDSTYSLSSTLELSKADYEKHKVYACEVTHQGLSSPVTKSFNRGEC

SEQ ID NO: 24 - protein, 57 aa, Artificial Sequence
  anti-CD47 IgG1 LC Constant Region
ELKSGTASVVCWLNNFYPREAKVQWKVDNALQSGNSEESVTEQDSKDSTYSLSSTLE

SEQ ID NO: 25 - protein, 330 aa, Artificial Sequence
  anti-CD47 IgG1 HC Constant Region
ASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 26 - protein, 220 aa, Artificial Sequence
  anti-CD47 IgG1 HC Constant Region
KSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTW

SEQ ID NO: 27 - protein, 107 aa, Artificial Sequence
  anti-CD20 IgG1 LC Constant Region
RTVAAPSVAIFPPSDERLKSGTASVVCVLNNFYPREAKVQWKVDNALQSGNSQESVTEQDSKDSTYSLSSRLTLSKADYEKHKVYACEVTHQGLSSPVTKSFNRGEC

SEQ ID NO: 28 - protein, 63 aa, Artificial Sequence
  anti-CD20 IgG1 LC Constant Region
AIFPPSDERLKSGTASVVCVLNNFYPREAKVQWKVDNALQSGNSQESVTEQDSKDSTYSLSSR

SEQ ID NO: 29 - protein, 330 aa, Artificial Sequence
  anti-CD20 IgG1 HC Constant Region
ASTKGPSVFPLAPSSKSTSGGTAWLGCEVTDYFPEPVTVSWNSGALTSGVHTFPAVLESSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 30 - protein, 267 aa, Artificial Sequence
  anti-CD20 IgG1 HC Constant Region portion
WLGCEVTDYFPEPVTVSWNSGALTSGVHTFPAVLESSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALV

SEQ ID NO: 31 - protein, 11 aa, Artificial Sequence
  90002 VL CDR1
KASQDIHRYLS

SEQ ID NO: 32 - protein, 7 aa, Artificial Sequence
  90002 VL CDR2
RANRLVS

SEQ ID NO: 33 - protein, 9 aa, Artificial Sequence
  90002 VL CDR3
LQYDEFPYT

SEQ ID NO: 34 - protein, 5 aa, Artificial Sequence
  90002 VH CDR1
DYYLH

SEQ ID NO: 35 - protein, 17 aa, Artificial Sequence
  90002 VH CDR2
WIDPDQGDTEYAQKFQD

SEQ ID NO: 36 - protein, 12 aa, Artificial Sequence
  90002 VH CDR3
AAYGSSSYPMDY

SEQ ID NO: 37 - protein, 10 aa, Artificial Sequence
  Rituximab CD20 VL CDR1
RASSSVSYIH

SEQ ID NO: 38 - protein, 7 aa, Artificial Sequence
  Rituximab CD20 VL CDR2
ATSNLAS

SEQ ID NO: 39 - protein, 9 aa, Artificial Sequence
  Rituximab CD20 VL CDR3
QQWTSNPPT

SEQ ID NO: 40 - protein, 5 aa, Artificial Sequence
  Rituximab CD20 VH CDR1
SYNMH

SEQ ID NO: 41 - protein, 17 aa, Artificial Sequence
  Rituximab CD20 VH CDR2
AIYPGNGDTSYNQKFKG

SEQ ID NO: 42 - protein, 12 aa, Artificial Sequence
  Rituximab CD20 VH CDR3
STYYGGDWYFNV

SEQ ID NO: 43 - protein, 11 aa, Artificial Sequence
  TPP-1361 CD47 VL CDR1
QASQDIHRYLS

SEQ ID NO: 44 - protein, 7 aa, Artificial Sequence
  TPP-1361 CD47 VL CDR2
RESRFVD

SEQ ID NO: 45 - protein, 9 aa, Artificial Sequence
  TPP-1361 CD47 VL CDR3
LQYDEFPYT

SEQ ID NO: 46 - protein, 5 aa, Artificial Sequence
  TPP-1361 CD47 VH CDR1
DYYLH

SEQ ID NO: 47 - protein, 17 aa, Artificial Sequence
  TPP-1361 CD47 VH CDR2
WIDPDQGDTYYAQKFQG

SEQ ID NO: 48 - protein, 11 aa, Artificial Sequence
  TPP-1361 CD47 VH CDR3
AYGESSYPMDY

SEQ ID NO: 49 - protein, 11 aa, Artificial Sequence
  TPP-1360 CD47 VL CDR1
RASQDIHRYLS

SEQ ID NO: 50 - protein, 7 aa, Artificial Sequence
  TPP-1360 CD47 VL CDR2
RESRFVD

SEQ ID NO: 51 - protein, 9 aa, Artificial Sequence
  TPP-1360 CD47 VL CDR3
LQYDEFPYT

SEQ ID NO: 52 - protein, 5 aa, Artificial Sequence
  TPP-1360 CD47 VH CDR1
DYYLH

SEQ ID NO: 53 - protein, 17 aa, Artificial Sequence
  TPP-1360 CD47 VH CDR2
WIDPDQGDTYYAQKFQG

SEQ ID NO: 54 - protein, 11 aa, Artificial Sequence
  TPP-1360 CD47 VH CDR3
AYGESSYPMDY

SEQ ID NO: 55 - protein, 11 aa, Artificial Sequence
  TPP-1367 CD47 VL CDR1
RASQDIHRYLS

SEQ ID NO: 56 - protein, 7 aa, Artificial Sequence
  TPP-1367 CD47 VL CDR2
RANRLVS

SEQ ID NO: 57 - protein, 9 aa, Artificial Sequence
  TPP-1367 CD47 VL CDR3
LQYDEFPYT

SEQ ID NO: 58 - protein, 5 aa, Artificial Sequence
  TPP-1367 CD47 VH CDR1
DYYLH

SEQ ID NO: 59 - protein, 17 aa, Artificial Sequence
  TPP-1367 CD47 VH CDR2
WIDPDQGDTYYAQKFQG

SEQ ID NO: 60 - protein, 11 aa, Artificial Sequenc
  TPP-1367 CD47 VH CDR3
AYGESSYPMDY

SEQ ID NO: 61 - protein, 11 aa, Artificial Sequence
  TPP-1362 CD47 VL CDR1
RASQGISSWLA

SEQ ID NO: 62 - protein, 7 aa, Artificial Sequence
  TPP-1362 CD47 VL CDR2
AASVLES

SEQ ID NO: 63 - protein, 9 aa, Artificial Sequence
  TPP-1362 CD47 VL CDR3
QQANSFPYT

SEQ ID NO: 64 - protein, 5 aa, Artificial Sequence
  TPP-1362 CD47 VH CDR1
NFVMS

SEQ ID NO: 65 - protein, 17 aa, Artificial Sequence
  TPP-1362 CD47 VH CDR2
TISGSGGSTYYADSVKG

SEQ ID NO: 66 - protein, 8 aa, Artificial Sequence
  TPP-1362 CD47 VH CDR3
HYILRYFD

SEQ ID NO: 67 - protein, 107 aa, Artificial Sequence
  TPP-1362 CD47 VL
DIQMTQSPSSVSASVGDRVTITCRASQGISSWLAWYQQKPGKAPKLLIYAASVLESGVPSRFSGSGSGTDFTLTISSLQPEDFATYYCQQANSFPYTFGQGTKLEIK

SEQ ID NO: 68 - protein, 117 aa, Artificial Sequence
  TPP-1362 CD47 VH
EVQLLESGGGLVQPGGSLRLSCAASGFTFPNFVMSWVRQAPGKGLEWVSTISGSGGSTYYADSVKGRFTISRDNSKNMLYLQMNSLRAEDTAVYYCAKHYILRYFDWLAGTLVTVSS

SEQ ID NO: 69 - protein, 214 aa, Artificial Sequence
  TPP-1362 CD47 WHOLE LC
DIQMTQSPSSVSASVGDRVTITCRASQGISSWLAWYQQKPGKAPKLLIYAASVLESGVPSRFSGSGSGTDFTLTISSLQPEDFATYYCQQANSFPYTFGQGTKLEIKRTVAAPSVFIFPPSDEELKSGTASVVCWLNNFYPREAKVQWKVDNALQSGNSEESVTEQDSKDSTYSLSSTLELSKADYEKHKVYACEVTHQGLSSPVTKSFNRGEC

SEQ ID NO: 70 - protein, 447 aa, Artificial Sequence
  TPP-1362 CD47 WHOLE HC
EVQLLESGGGLVQPGGSLRLSCAASGFTFPNFVMSWVRQAPGKGLEWVSTISGSGGSTYYADSVKGRFTISRDNSKNMLYLQMNSLRAEDTAVYYCAKHYILRYFDWLAGTLVTVSSASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 71 - protein, 107 aa, Artificial Sequence; identical to SEQ ID NO: 5
  408_437 VL

SEQ ID NO: 72 - protein, 120 aa, Artificial Sequence
  408_437 VH
QMQLVQSGAEVKKPGSSVKVSCKASGFNIKDYYLHWVRQAPGKGLEWMGWIDPDQGDTEYAQKFQGRVTITRDRSTSTAYMELRSLRAEDTAVYYCNAAYGSSSYPMDYWGQGTLVTVSS

(19) United States
(12) Patent Application Publication
(10) Pub. No.: US 2023/0365681 A1
(43) Pub. Date: Nov. 16, 2023

(54) BISPECIFIC ANTIBODY TREATMENT OF LYMPHOID MALIGNANT NEOPLASM CONDITIONS

(71) Applicant: CELGENE CORPORATION, Summit, NJ (US)

(72) Inventors: Haralambos HADJIVASSILIOU, Summit, NJ (US); Dan ZHU, Summit, NJ (US); Jeonghoon SUN, Summit, NJ (US); Sharmistha ACHARYA, Summit, NJ (US); Jeffrey JOHNSON, Summit, NJ (US); Kandasamy HARIHARAN, Summit, NJ (US); Ho CHO, Summit, NJ (US)

(21) Appl. No.: 18/248,205

(22) PCT Filed: Oct. 5, 2021
(86) PCT No.: PCT/US2021/071718

§ 371 Date: Apr. 6, 2023

Related U.S. Application Data
(60) Provisional application No. 63/088,879, filed on Oct. 7, 2020.

(51) Int. Cl. C07K 16/28; A61P 35/00; A61K 39/00

(52) U.S. Cl. CPC C07K 16/2803; C07K 16/2887; A61P 35/00; C07K 2317/50; C07K 2317/92; A61K 2039/505; C07K 2317/33; C07K 2317/31; C07K 2317/76; C07K 2317/55

(57) ABSTRACT

Provided is a method for the treatment of a lymphoid malignant neoplasm in a subject in need thereof comprising administering to the subject an effective amount of a bispecific antibody which comprises a Fab portion that binds CD47 and a Fab portion that binds CD20. In some embodiments, the lymphoid malignant neoplasm is Non-Hodgkin's Lymphoma (NHL), follicular lymphoma (FL), diffuse large B-cell lymphoma (DLBCL), marginal zone lymphoma (MZL), mantle cell lymphoma (MCL), or primary mediastinal B-cell lymphoma. In certain embodiments, the lymphoid malignant neoplasm is NHL.

### CROSS REFERENCE TO RELATED APPLICATIONS

This application is entitled to priority under 35 U.S.C. § 119(e) to U.S. Provisional Application No. 63/088,879, filed Oct. 7, 2020, which is hereby incorporated by reference in its entirety.

### FIELD

Methods of treatment and control of lymphoid malignant neoplasm conditions are provided which employ distinct anti-CD20/anti-CD47 bispecific antibody species. The methods also provide an unmet medical need for patients relapsed or refractory from current anticancer therapy or wherein no other approved conventional therapy exists for the condition.

### SEQUENCE LISTING

This application contains a Sequence Listing which is filed herewith in ASCII format via EFS-Web and is hereby incorporated by reference in its entirety. The ASCII copy, created on Sep. 27, 2021, is named 298068-00369_Sequence_Listing.txt and is 77,634 bytes.

### BACKGROUND

Non-Hodgkin lymphoma (NHL) is a heterogeneous group of lymphoid malignant neoplasms with diverse biological and clinical presentations. About 85 to 90% of NHL is derived from B cells and about 65% of all NHL falls into two subtypes, follicular lymphoma (FL) and diffuse large B-cell lymphoma (DLBCL). Non-Hodgkin lymphoma can be divided into 2 prognostic groups: the indolent lymphoma (slowly growing with waxing and waning lymphadenopathy for years) and the aggressive lymphoma (rapidly growing and resulting in death within a few weeks if not treated). It is estimated that there will be 77,240 new cases and 19,940 deaths from Non-Hodgkin lymphoma (NHL) in the United States (US) in 2020 (Siegel R L, Miller K D, Jemal A. Cancer statistics, 2020. CA Cancer J Clin. 2020; 70(1):7-30).

The most common aggressive lymphoma is DLBCL accounting for 30 to 40% of all NHL (Li S, Young K H, Medeiros U. Diffuse large B-cell lymphoma. Pathology. 2018 January; 50(1):74-87). Other aggressive lymphomas include but are not limited to high grade B-cell lymphoma (HGBCL), so-called double hit (DHL) or triple hit lymphoma (THL), mantle cell lymphoma (MCL), primary mediastinal large B-cell lymphoma (PMBCL), and follicular lymphoma grade 3b (FL3B). Prognosis and therapy of other aggressive lymphoma subtypes are similar to DLBCL.

Follicular lymphoma (FL) is the most common subtype of indolent NHL, accounting for approximately 22% of newly diagnosed NHL cases. Involved-site radiotherapy remains the standard of care for early-stage FL patients with limited sites of disease. For patients who require systemic therapy, rituximab in conjunction with chemotherapy is often employed as frontline therapy. There is no standard treatment for relapsed or refractory FL patients. Alternate first-line chemoimmunotherapy regimens are frequently utilized as second-line therapy along with options such as single agent rituximab, lenalidomide in combination with rituximab, or Phosphoinositide 3-Kinase (PI3K) inhibitors. Patients who fail to respond to a rituximab-containing regimen and have relapsed on or are refractory to additional therapies have limited treatment options and a poor prognosis. Novel agents alone or in combination with conventional therapies has led to significant improvements in the clinical outcomes of FL patients. However, despite these recent advancements, FL remains an incurable disease with multiple relapses and shorter duration of response to subsequent treatment (Rivas-Delgado A, Magnano L, Moreno-Velazquez M, et al. Progression-free survival shortens after each relapse in patients with follicular lymphoma treated in the rituximab era. Hematological Oncology. 2017; 35(S2):360-361).

Approximately 50% of newly diagnosed patients with DLBCL, for example, can be cured with first-line R-CHOP immunochemotherapy (rituximab, cyclophosphamide, doxorubicin, vincristine and prednisone). However, approximately half of the patients treated with R-CHOP will relapse, mostly within the first 2 years after therapy (Coiffier B, et al. Long-term outcome of patients in the LNH-98.5 trial, the first randomized study comparing rituximab-CHOP to standard CHOP chemotherapy in DLBCL patients: a study by the Groupe d'Etudes des Lymphomes de l'Adulte. Blood. 2010 Sep. 23; 116(12):2040-5; Vitolo U, et al. Obinutuzumab or rituximab plus cyclophosphamide, doxorubicin, vincristine, and prednisone in previously untreated diffuse large B-cell lymphoma. J Clin Oncol. 2017 Nov. 1; 35(31):3529-37). The standard second-line therapy is salvage high-dose immuno-chemotherapy followed by autologous stem-cell transplantation (ASCT) for relapsed or refractory (R/R) DLBCL. However, only around half of the patients are eligible for ASCT, but 50% of those patients still experience relapse within 3 years after auto-HSCT (Bachanova V, Perales M A, Abramson J S. Modern management of relapsed and refractory aggressive B-cell lymphoma: A perspective on the current treatment landscape and patient selection for CAR T-cell therapy. Blood Rev. 2020; 40:100640). The other half of the R/R patients will not be transplant-eligible due to inadequate response to salvage therapy or medical comorbidities. These patients can be treated with less intensive second-line therapies, but the intent is usually palliative and there is no single regimen established for this population. Recent advances in CD19-targeted chimeric antigen receptor CAR-T cell therapies led to approval of Kymriah™ (tisagenlecleucel) and Yescarta® (axicabtagene ciloleucel) for treatment of R/R DLBCL, HGBCL, DLBCL arising from FL and PMBCL after ≥2 prior lines of therapy. Kymriah™'s JULIET study and Yescarta®'s ZUMA-1 study reported CR rates of 40% and 58%, respectively (Locke F L, Ghobadi A, Jacobson C A, et al. Long-term safety and activity of axicabtagene ciloleucel in refractory large B-cell lymphoma (ZUMA-1): a single-arm, multicentre, phase 1-2 trial. Lancet Oncol. 2019; 20(1):31-42; Bachanova V, Westin J, Tam C et al. Correlative analyses of cytokine release syndrome and neurological events in tisagenlecleucel-treated relapsed/refractory diffuse large B-cell lymphoma patients. Clinical Lymphoma, Myeloma & Leukemia. 2019; 19:5251-252). In addition, Transcend-NHL-001 study evaluating Lisocabtagene Maraleucel (liso-cel) in R/R large B cell lymphomas reported a CR rate of 53% (Abramson J S, et al. Pivotal Safety and Efficacy Results from Transcend NHL 001, a Multicenter Phase 1 Study of Lisocabtagene Maraleucel (liso-cel) in Relapsed/Refractory (R/R) Large B Cell Lymphomas. Blood 2019; 134 (Supplement_1): 241). Despite encouraging results of CAR-T cell therapies, approximately half of these patients do not respond well and remain as an unmet need population.

### SUMMARY

Provided herein is a method for the treatment of a lymphoid malignant neoplasm in a subject in need thereof comprising administering to the subject an effective amount of a bispecific antibody which comprises:
i) a Fab portion that binds CD47 comprising a light chain variable region (VL) comprising a VL CDR1 comprising the amino acid sequence QASQDIHRYLS (SEQ ID NO:43) or RASQDIHRYLS (SEQ ID NO:49); a VL CDR2 comprising the amino acid sequence RESRFVD (SEQ ID NO:50) or RANRLVS (SEQ ID NO:56); and a VL CDR3 comprising the amino acid sequence LQYDEFPYT (SEQ ID NO:51); and a heavy chain variable region (VH) comprising a VH CDR1 comprising the amino acid sequence DYYLH (SEQ ID NO:52); a VH CDR2 comprising the amino acid sequence WIDPDQGDTYYAQKFQG (SEQ ID NO:53); and a VH CDR3 comprising the amino acid sequence AYGESSYPMDY (SEQ ID NO:54); and,
ii) a Fab portion that binds CD20 comprising a light chain variable region (VL) region comprising a VL CDR1 comprising the amino acid sequence RASSSVSYIH (SEQ ID NO:37), a VL CDRs comprising the amino acid sequence ATSNLAS (SEQ ID NO:38), and a VL CDR3 comprising the amino acid sequence QQWTSNPPT (SEQ ID NO:39); and a heavy chain variable region (VH) region comprising a VH CDR1 comprising the amino acid sequence SYNMH (SEQ ID NO:40), a VH CDR2 comprising the amino acid sequence AIYPGNGDTSYNQKFKG (SEQ ID NO:41), and STYYGGDWYFNV (SEQ ID NO:42).

In specific embodiments, provided herein is a method for the treatment of a lymphoid malignant neoplasm in a subject wherein the Fab portion that binds CD47 comprises a light chain variable region (VL) comprising, or consisting of, the amino acid sequence of SEQ ID NO:1, SEQ ID NO:3, or SEQ ID NO:5; and a heavy chain variable region (VH) comprising, or consisting of, SEQ ID NO:2, SEQ ID NO:4, or SEQ ID NO:6.

Further provided herein is a method for the treatment of a lymphoid malignant neoplasm in a subject wherein the bispecific antibody comprises an anti-CD47 light chain comprising, or consisting of, the amino acid sequence of SEQ ID NO:17, SEQ ID NO:19, and SEQ ID NO:21; and, an anti-CD47 heavy chain comprising, or consisting of, the amino acid sequence of SEQ ID NO:18, SEQ ID NO:20, or SEQ ID NO:22.

Further provided herein is a method for the treatment of a lymphoid malignant neoplasm in a subject wherein the bispecific antibody comprises an anti-CD47 heavy chain, e.g., comprising, or consisting of, the amino acid sequence of SEQ ID NO:18, SEQ ID NO:20, or SEQ ID NO:22, but wherein said anti-CD47 heavy chain lacks the C-terminal lysine.

Further provided herein is a method for the treatment of a lymphoid malignant neoplasm in a subject wherein the bispecific IgG1 antibody comprises an anti-CD20 light chain, e.g., comprising the amino acid sequence of SEQ ID NO:15 and an anti-CD20 heavy chain comprising the amino acid sequence of SEQ ID NO:16.

Further provided herein is a method for the treatment of a lymphoid malignant neoplasm in a subject wherein the bispecific antibody comprises an anti-CD20 heavy chain, e.g., comprising, or consisting of, the amino acid sequence of SEQ ID NO:16, but wherein said anti-CD20 heavy chain lacks the C-terminal lysine.

Further provided herein is a method for the treatment of a lymphoid malignant neoplasm in a subject wherein the bispecific antibody comprises an anti-CD47 light chain comprising, or consisting of, the amino acid sequence of SEQ ID NO:19; and an anti-CD47 heavy chain comprising, or consisting of, the amino acid sequence of SEQ ID NO:20.

Further provided herein is a method for the treatment of a lymphoid malignant neoplasm in a subject wherein the bispecific antibody comprises an anti-CD47 heavy chain, e.g., comprising, or consisting of, the amino acid sequence of SEQ ID NO:20, but wherein said anti-CD47 heavy chain lacks the C-terminal lysine.

In specific embodiments of the methods of treatment provided herein, the lymphoid malignant neoplasm is Non-Hodgkin's Lymphoma (NHL), follicular lymphoma (FL), diffuse large B-cell lymphoma (DLBCL), marginal zone lymphoma (MZL), mantle cell lymphoma (MCL), or primary mediastinal B-cell lymphoma.

In some embodiments of any of the embodiments provided herein, the subject has relapsed or refractory lymphoid malignant neoplasm. In further embodiments, the lymphoid malignant neoplasm has progressed on standard anticancer therapy. In yet further embodiments of any of the embodiments provided herein, no other approved conventional therapy exists for said subject having said lymphoid malignant neoplasm.

In some embodiments of the methods provided herein, the lymphoid malignant neoplasm is NHL. In further embodiments, the lymphoid malignant neoplasm is relapsed or refractory NHL.

In some embodiments of the methods provided herein, the lymphoid malignant neoplasm is follicular lymphoma.

In some embodiments of the methods provided herein, the lymphoid malignant neoplasm is diffuse large B-cell lymphoma.

In some embodiments of the methods provided herein, the lymphoid malignant neoplasm is marginal zone lymphoma.

In some embodiments of the methods provided herein, the lymphoid malignant neoplasm is mantle cell lymphoma.

In some embodiments of the methods provided herein, the lymphoid malignant neoplasm is primary mediastinal B-cell lymphoma.

In a specific embodiment of the method of the treatment of a lymphoid malignant neoplasm in a subject provided herein, the lymphoid malignant neoplasm is NHL, and the subject has at least one nodal lesion >1.5 cm in its longest diameter, or at least one extranodal lesion >1.0 cm in its longer diameter, on cross sectional imaging by CT or MRI as defined by Lugano criteria.

In specific embodiments of the methods for the treatment of a lymphoid malignant neoplasm in a subject provided herein, the subject has one or more of:
a. absolute neutrophil count (ANC) ≥1.0×109/L without growth factor support for 7 days (14 days if on pegfilgrastim);
b. hemoglobin (Hgb) ≥8 g/dL without transfusion for 14 days;
c. platelets (plt) ≥75×109/L without transfusion for 7 days;
d. aspartate aminotransferase (AST/SGOT) and alanine aminotransferase (ALT/SGPT)≤2.5× Upper Limit of Normal (ULN) or ≤5.0×ULN if tumor is present in the liver;
e. serum bilirubin ≤1.5×ULN;
f. estimated serum creatinine clearance of ≥45 mL/min using the Cockcroft-Gault equation or measured creatinine clearance using 24-hour urine collection; and/or
g. international normalized ratio (INR) <1.5×ULN and partial thromboplastin time (PTT) <1.5×ULN.

In other specific embodiments of the method for the treatment of a lymphoid malignant neoplasm in a subject provided herein, the subject:
a. does not have Burkitt's or lymphoblastic lymphoma;
b. does not have chronic lymphocytic leukemia (CLL) or small lymphocytic lymphoma (SLL) including Richter's transformation;
c. does not have cancer with symptomatic central nervous system (CNS) involvement;
d. is not on chronic systemic immunosuppressive therapy or corticosteroids exceeding a total dose of 140 mg within 14 days of said administration;
e. does not have clinically significant graft-versus-host disease (GVHD);
f. has no history of class III or IV congestive heart failure (CHF) or severe non ischemic cardiomyopathy, unstable angina, myocardial infarction, or ventricular arrhythmia within the previous 6 months;
g. does not have inadequate cardiac function, defined as left ventricular ejection fraction (LVEF) <45% as assessed by echocardiogram (ECHO) or multiple uptake gated acquisition (MUGA) scan performed within 30 days of said administration;
h. has not received prior investigational therapy directed at CD47 or SIRPα;
i. has not had treatment with CAR-T therapy ≤4 weeks prior to said administration;
j. has not had prior systemic cancer-directed treatments or investigational modalities ≤5 half-lives or 4 weeks prior to said administration, whichever is shorter;
k. has not had major surgery ≤2 weeks prior to starting TPP-1360, and wherein said subject has recovered from any clinically significant effects of any recent surgery;
l. has not had autologous stem cell transplant ≤3 months prior to said administration;
m. has not had allogeneic stem cell transplant with either standard or reduced intensity conditioning ≤6 months prior to said administration;
n. does not have diagnosed primary immune deficiency disease;
o. does not have diagnosed active human immunodeficiency virus (HIV) infection; except where said subject has well controlled HIV with CD4+ T-cell (CD4+) counts ≥350 cells/uL without opportunistic infection within 12 months prior to said administration;
p. does not have active hepatitis B virus (HBV) or hepatitis C virus (HCV) infection;
q. is not on ongoing treatment with chronic, therapeutic dosing of an anti-coagulant;
r. has no history of autoimmune hemolytic anemia or autoimmune thrombocytopenia;
s. has no history of concurrent second cancers requiring active, ongoing systemic treatment; and/or
t. has not had a live virus vaccine within at least 4 weeks prior to said administration.

### BRIEF DESCRIPTION OF THE FIGURES

FIG. 1 is a schematic illustration of certain attributes of bispecific entities described herein engineered to overcome the challenge of ubiquitous CD47 expression including low affinity binding without avidity to CD47; minimal binding to normal cells, i.e., no tissue sink (that is, no spurious binding of the bispecific antibody to normal CD47-expressing cells in the absence of CD20); high affinity selective avidity binding to CD20 which results in selective binding to tumor cells. TAA: Tumor Associated Antigen.

FIG. 2 illustrates an example bispecific entity architecture, protein engineering features, and several biopharmacological attributes.

FIGS. 3A-3C show that example species bispecific entities described herein induce macrophage-mediated phagocytosis of CD20+ CD47+ OCI-Ly3 NHL cells. FIGS. 3A-3B are graphs that show the percentage of phagocytic macrophages in view of antibody concentration. FIG. 3C is a table showing KD and EC50 values for bispecific species described herein.

FIGS. 4A-4C show that example bispecific entities, CD47×CD20 IgG1 species, described herein demonstrate CDC function. FIGS. 4A-4B are graphs that show CDC in view of antibody concentration. FIG. 4C is a table showing average EC50 values for TPP-1360, TPP-1362 and rituximab.

FIGS. 5A-5C show that example bispecific entities, CD47×CD20 IgG1 species, described herein demonstrate potent ADCC function in CD20 high NHL cells, i.e., significantly higher than rituximab. FIGS. 5A-5B are graphs that show cytotoxicity in view of antibody concentration. FIG. 5C is a table showing CD20/CD47 Ratio.

FIG. 6 illustrates example architecture of bispecific entities described herein as well as features of certain examples.

FIG. 7 shows an example species bispecific entity described herein, TPP-1360, that substantially shifted the binding signal to B-cells and rather weakly to T cells, monocytes, and NK cells, with minimal or no binding to platelets or red blood cells as compared to binding of TPP-23 (408_437 Fab (VL: SEQ ID NO:71; VH: SEQ ID NO:72) with IgG1), thereby illustrating selective binding to B-cells in human whole blood.

FIG. 8 illustrates an example species bispecific entity described herein, TPP-1360, demonstrated to selectively bind CD47⁺/CD20⁺ Raji Cells but not CD47⁺/CD20⁻ human red blood cells (RBCs).

FIG. 9 shows that, in a co-culture of Raji cells and human RBCs, an example species bispecific entity described herein, TPP-1360, displays dose-dependent binding to CD47⁺/CD20⁺ Raji cells but no binding to human RBCs, even at concentration as high as 1 mg/mL.

FIG. 10 illustrates that TPP-1360, for example, potently and completely blocks recombinant human SIRPα-Fc binding to human CD47 expressed on the surface of CD20⁺/CD47⁺ lymphoma cell line OCI-Ly3.

FIG. 11 illustrates that TPP-1360, for example, potently and completely blocks recombinant human SIRPα-Fc binding to human CD47 expressed on the surface of CD20⁺/CD47⁺ lymphoma cell line Raji.

FIG. 12 illustrates that treatment with TPP-1360, for example, induced macrophage-mediated phagocytosis of the CD20⁺ malignant B cell line, Raji.

FIG. 13 illustrates that treatment with TPP-1360, for example, induced macrophage-mediated phagocytosis of the CD20⁺ malignant B cell line, OCI-Ly3.

FIG. 14 illustrates that treatment with TPP-1360, for example, induced macrophage-mediated phagocytosis of the CD20⁺ malignant B cell line, REC-1.

FIG. 15 illustrates that treatment with TPP-1360, for example, induced macrophage-mediated phagocytosis of the CD20⁺ malignant B cell line, RIVA.

FIG. 16 shows that treatment with TPP-1360, for example, triggered significantly more efficient phagocytosis than rituximab in Raji and OCI-Ly3 cells, likely due to the concomitant blockade of the SIRPα-CD47 interaction and the engagement of activating receptors, such as FcγRs, by TPP-1360.

FIG. 17 shows binding of rituximab and bispecific antibodies such as TPP-1360, for example, to Raji cells (CD20⁺/CD47⁺) as measured by surface plasmon resonance (SPR).

FIG. 18 illustrates that TPP-1360 and TPP-1362, for example, potently and completely block recombinant human SIRPα binding to human CD47 expressed on the surface of CD20₊/CD47⁺ lymphoma cell line OCI-Ly3. Rituximab was found to have no effect on SIRPα binding.

FIG. 19 is a schematic illustration of modes of action of bispecific entities described herein.

FIG. 20 shows that TPP-1360 single agent phagocytosis induction activity in CD20⁺/CD47⁺ lymphoma cell line OCI-Ly3 is comparable to that of rituximab combined with anti-CD47 IgG4PE. CD=cluster of differentiation; IgG=immunoglobulin G1; IgG4PE=immunoglobulin G4 with serine to proline substitution at position 228 and leucine to glutamic acid substitution at position 235; RSV=respiratory syncytial virus; TPP-23=bivalent antibody with non-attenuated affinity to CD47; TPP-356=an anti-CD47 IgG4PE antibody with non-attenuated affinity to CD47.

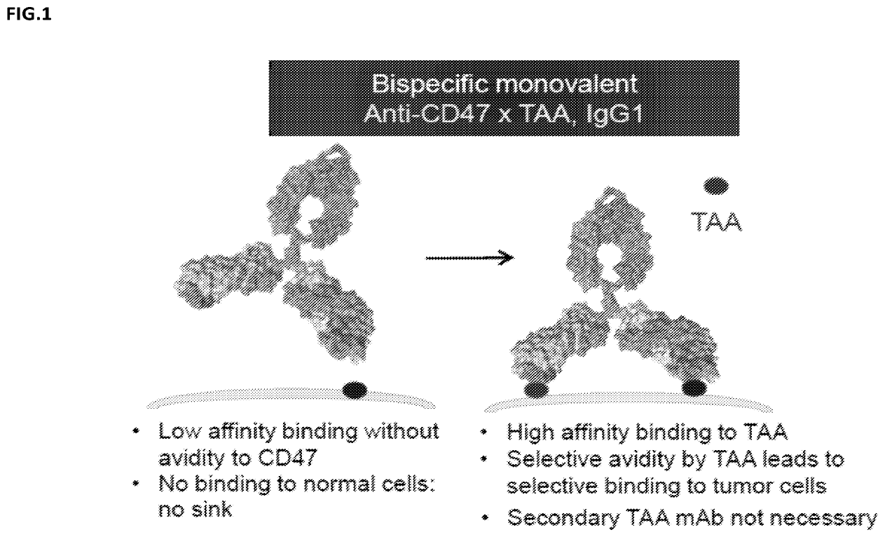

### DETAILED DESCRIPTION

Unless defined otherwise, all technical and scientific terms used herein have the same meaning as is commonly understood by one of skill in the art to which this disclosure belongs. All publications and patents referred to herein are incorporated by reference.

Cluster of differentiation (CD47) is overexpressed in NHL cells and has shown correlation with worse prognosis in multiple NHL subtypes. The inhibitory CD47-SIRPα ligand-receptor pair has been identified as an important, but not universal, innate immune checkpoint regulator in the homeostatic clearance by macrophages. When CD47 on lymphocytes bind to SIRPα on macrophages, this triggers the “don't eat me” signal to the macrophage, preventing its robust phagocytic ability.

Macrophages express SIRPα which interacts with CD47, a ubiquitously expressed protein that mediates a “don't eat me” signal that functions to inhibit phagocytosis. Cancer cells have evolved to hijack this interaction by upregulating the expression of CD47 on their cell surface, thus counterbalancing pro-phagocytic signals and increasing the chances of evading innate immune surveillance.

As used herein, the articles “a” and “an” may refer to one or to more than one (e.g. to at least one) of the grammatical object of the article.

As used herein, “about” may generally refer to an acceptable degree of error for the quantity measured given the nature or precision of the measurements. Example degrees of error are within 5% of a given value or range of values.

Embodiments described herein as “comprising” one or more features may also be considered as disclosure of the corresponding embodiments “consisting of” and/or “consisting essentially of” such features.

The term “lymphoid malignant neoplasm,” as used herein, refers to a pathological condition manifested by malignant CD20+ lymphoid cells at various stages of differentiation, including but not limited to Non-Hodgkin lymphoma (NHL), relapsed or refractory (R/R) non-Hodgkin's lymphoma (NHL), diffuse large B-cell lymphoma (DLBCL), relapsed or refractory (R/R) DLBCL, follicular lymphoma (FL), high grade B-cell lymphoma (HGBCL), double hit (DHL) or triple hit lymphoma (THL), mantle cell lymphoma (MCL), primary mediastinal large B-cell lymphoma (PMBCL), follicular lymphoma grade 3b (FL3B); as well as, R/R DLBCL, HGBCL, and DLBCL arising from FL or PMBCL.

Bispecific anti-CD47/anti-CD20 heterodimeric IgG1 entities described herein are developed, for example, as intravenous (IV) injectable treatment for CD20+ B-cell lymphoma patients, and particularly conditions refractory and/or resistant to current therapies.

The terms “tumor” and “tumor cell” as used herein broadly refers to CD20+ cancer cells undergoing aberrant proliferation which manifest a lymphoid malignant neoplasm.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Minor variations in the amino acid sequences of antibodies provided herein are contemplated as being encompassed by the present disclosure, providing that the variations in the amino acid sequence(s) maintain at least 75%, at least 80%, at least 90%, at least 95%, or at least 98 or 99% sequence homology or identity to the sequence of an antibody or antigen-binding fragment thereof provided herein.

Antibodies provided herein may include variants in which amino acid residues from one species are substituted for the corresponding residue in another species, either at the conserved or non-conserved positions. In one embodiment, amino acid residues at non-conserved positions are substituted with conservative or non-conservative residues. In particular, conservative amino acid replacements are contemplated.

As used herein, a “conservative amino acid substitution” refers to one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in an antibody provided herein does not exclude other forms of variant, for example polymorphic variants, interspecies homologs, and alleles.

As used herein, “non-conservative amino acid substitutions” include those in which (i) a residue having an electropositive side chain (e.g., arginine, histidine or lysine) is substituted for, or by, an electronegative residue (e.g., glutamate or aspartate), (ii) a hydrophilic residue (e.g., serine or threonine) is substituted for, or by, a hydrophobic residue (e.g., alanine, leucine, isoleucine, phenylalanine or valine), (iii) a cysteine or proline is substituted for, or by, any other residue, or (iv) a residue having a bulky hydrophobic or aromatic side chain (e.g., valine, histidine, isoleucine or tryptophan) is substituted for, or by, one having a smaller side chain (e.g., alanine or serine) or no side chain (e.g., glycine).

The terms “antibody” and “antibodies”, as used herein, refers to conventional isotypes and monospecific formats as well as multivalent antibodies including but not limited to current bispecific entity formats known in the art as well as bispecific antibodies including but not limited to formats otherwise described herein.

A typical antibody comprises at least two “light chains” (LC) and two “heavy chains” (HC). The light chains and heavy chains of such antibodies are polypeptides consisting of several domains. Each heavy chain comprises a heavy chain variable region (abbreviated herein as “VH”) and a heavy chain constant region (abbreviated herein as “CH”). The heavy chain constant region comprises the heavy chain constant domains CH1, CH2 and CH3 (antibody classes IgA, IgD, and IgG) and optionally the heavy chain constant domain CH4 (antibody classes IgE and IgM). Each light chain comprises a light chain variable domain (abbreviated herein as “VL”) and a light chain constant domain (abbreviated herein as “CL”). The variable regions VH and VL can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The “constant domains” of the heavy chain and of the light chain are not involved directly in binding of an antibody to a target, but exhibit various effector functions.

Binding between an antibody and its target antigen or epitope is mediated by the Complementarity Determining Regions (CDRs). The CDRs are regions of high sequence variability, located within the variable region of the antibody heavy chain and light chain, where they form the antigen-binding site. The CDRs are the main determinants of antigen specificity. Typically, the antibody heavy chain and light chain each comprise three CDRs which are arranged non-consecutively. The antibody heavy and light chain CDR3 regions play a particularly important role in the binding specificity/affinity of the antibodies provided herein and therefore provide a further aspect of the present disclosure.

Thus, the term “antigen binding fragment” as used herein incudes any naturally-occurring or artificially-constructed configuration of an antigen-binding polypeptide comprising one, two or three light chain CDRs, and/or one, two or three heavy chain CDRs, wherein the polypeptide is capable of binding to the antigen.

The sequence of a CDR may be identified by reference to any number system known in the art, for example, the Kabat system (Kabat, E. A., et al., Sequences of Proteins of Immunological Interest, 5th ed., Public Health Service, National Institutes of Health, Bethesda, M D (1991); the Chothia system (Chothia &, Lesk, “Canonical Structures for the Hypervariable Regions of Immunoglobulins,” J. Mol. Biol. 196, 901-917 (1987)); or the IMGT system (Lefranc et al., “IMGT Unique Numbering for Immunoglobulin and Cell Receptor Variable Domains and Ig superfamily V-like domains,” Dev. Comp. Immunol. 27, 55-77 (2003)). CDRs shown herein employ the boundaries, i.e., size, according to KABAT. Position numbering of antibody constant regions described and referred to herein are generally according to KABAT. However, numbering of anti-CD47 VL and VH regions described herein, i.e., antibody residue positions and substituted positions, begins with the N-terminal residue of each variable region, i.e., VL or VH, particularly with reference to SEQ ID NO:9 and SEQ ID NO:10, respectively.

“Bispecific entities described herein” generally refers to the functionally defined antibodies, bispecific elemental formats, elemental sequences, antibodies, and antibody species described herein.

Bispecific entities described herein selectively and safely target CD20+ lymphoid malignant neoplasm tumor cells with substantially no of binding to CD47 in peripheral tissues, RBCs, and platelets.

The term bispecific IgG1 antibody, as used herein, refers to an anti-CD47/anti-CD20 IgG1 1+1 heterodimer bispecific antibody.

### IgG1 Format

IgG1, as used herein, fundamentally refers to a whole IgG1 antibody composed of (i) one heavy chain (HC) and one light chain (LC), on one side; and, (ii) one heavy chain (HC) and one light chain (LC), on the other side.

### IgG1 1+1 Heterodimer Format

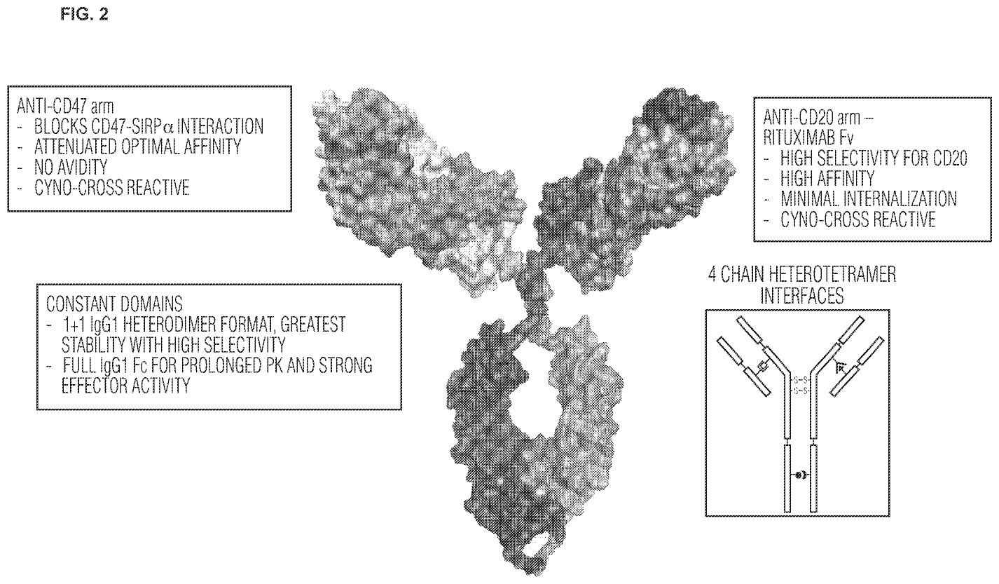

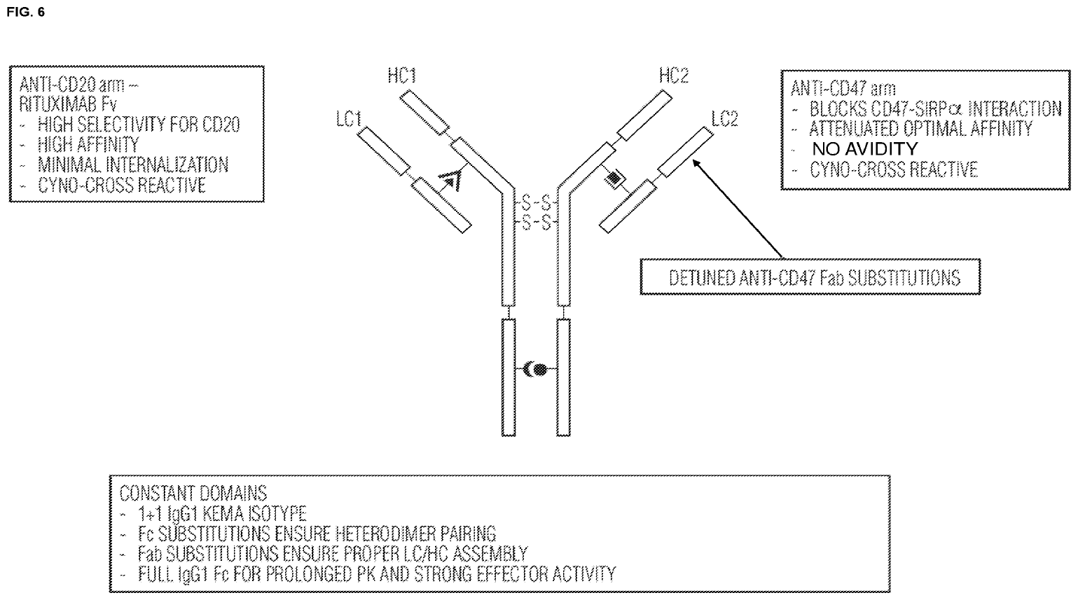

IgG1 or IgG1 1+1 heterodimer format, as used herein, fundamentally refers to a whole IgG1 antibody composed of (i) one heavy chain (HC) and one light chain (LC), on one side, from one source, i.e., anti-CD47; and, (ii) one heavy chain (HC) and one light chain (LC), on the other side, from another source, e.g., anti-CD20. See, e.g., FIG. 2 and FIG. 6.

Bispecific antibodies intended for employment in methods of the present disclosure are fundamentally native human IgG1 antibodies composed of, (A) one anti-CD47 IgG1 (monomeric) portion which contains one entire light chain (LC) and one entire heavy chain (HC), as well as (B) one anti-CD20 IgG1 (monomeric) portion which contains one entire light chain (LC) and one entire heavy chain (HC). The two monomers form a conventional dimeric IgG1 antibody wherein one arm (Fab₁) provides for attenuated binding of CD47 while the other arm (Fab₂) provides for affinity binding and avidity for CD20. FIG. 2 and FIG. 6 illustrate CD47×CD20 example architecture described herein and example protein engineering features.

The term “Fab portion” as used herein, refers to an antigen-binding fragment of an antibody, i.e., a region of an antibody that binds an antigen. As used herein it comprises one variable domain of each of a light and heavy chain (VL/VH).

CC-90002 is provided as a reference parental sequence and as a source of anti-CD47 elements for construction of some of the bispecific entities described herein. CC-90002 VL CDRs are SEQ ID NO:31 (CDRL1), SEQ ID NO:32 (CDRL2), and SEQ ID NO:33 (CDRL3). CC-90002 VH CDRs are SEQ ID NO:34 (CDRH1), SEQ ID NO:35 (CDRH2), and SEQ ID NO:36 (CDRH3). CC-90002 VL (SEQ ID NO:9) and VH (SEQ ID NO:10) are also provided for reference. CC-90002 VL fused to a native IgG1 LC constant region to form a whole LC for reference is provided as CC-90002 WHOLE LC/IgG1 (SEQ ID NO:11). CC-90002 VH fused to a native IgG1 HC constant region to form a whole HC for reference is provided as CC-90002 whole HC/IgG1 (SEQ ID NO:12). See, U.S. Pat. No. 9,045,541.

Antibodies of the present disclosure comprise VL and VH amino acid sequences derived from CC-90002, i.e., SEQ ID NO:9 and SEQ ID NO:10, respectively, wherein the binding affinity for CD47 is substantially attenuated, i.e., Fab portion that binds CD47 exhibits low affinity. Both VH and VL regions of CC-90002 were engineered to reduce immunogenicity, while retaining functionality for employment in bispecific entities described herein. Protein engineering was employed on both VH and VL regions of CC-90002 to reduce immunogenicity, while retaining functionality; and, particularly to detune affinity for CD47.

The anti-CD47 epitope was determined by solving the crystal structure of a non-detuned parental version of CC-90002 (TPP-23 (408_437 Fab (VL: SEQ ID NO:71; VH: SEQ ID NO:72) with IgG1)) in complex with the human CD47 extracellular domain at 2.4 Å resolution. See Example 3.

Bispecific antibodies provided herein generally comprise rituximab VL CDRs: SEQ ID NO:37 (CDRL1), SEQ ID NO:38 (CDRL2), and SEQ ID NO:39 (CDRL3); and, rituximab VH CDRs: SEQ ID NO:40 (CDRH1), SEQ ID NO:41 (CDRH2), and SEQ ID NO:42 (CDRH3), respectively. Bispecific antibodies provided herein generally comprise rituximab VL (SEQ ID NO:323) and rituximab VH (SEQ ID NO:324), respectively. Anti-CD20 LC (SEQ ID NO:15) is preferred for employment in construction of bispecific entities of the present disclosure. Anti-CD20 HC (SEQ ID NO:16) is preferred for employment in construction of bispecific entities of the present disclosure.

A CD47×CD20 bispecific program was initiated to identify therapeutic antibodies that are able to block human CD47 binding to SIRPα only on CD20 expressing lymphoid malignant neoplasm cells. Three (3) effective bispecific IgG1 antibodies resulting from that project provided herein bind with high affinity to CD20 while exhibiting a detuned affinity to CD47.

The variable heavy (VH) domains are fused to human IgG1 constant domains and the variable light (VL) domains are fused to human kappa constant domains. The IgG1 constant domains and the kappa constant domains contain amino acid substitutions to ensure the desired heterodimeric light chain (LC) and heavy chain (HC) pairing and the heterodimeric fragment crystallizable (Fc) pairing.

TPP-1360, for example, an immunoglobulin G1 (IgG1) bispecific antibody co-targeting CD47 and CD20, is designed to bind CD20 with high affinity and CD47 with optimally lowered (detuned) affinity. The detuned anti-CD47 arm, obtained through derivatization of Celgene's anti-CD47 monoclonal antibody CC-90002, was paired with the anti-CD20 arm from rituximab to form an IgG1 bispecific.

When bound to CD20 expressing cells, TPP-1360 also binds to CD47 to block the macrophage checkpoint inhibitor, SIRPα, while engaging in activating FcγRs expressed by macrophages to potentiate their engulfment and destruction of CD20 positive cells. Additionally, the anti-tumor activity of TPP-1360 includes engagement of activating FcRs on myeloid and NK cells to eliminate tumor cells via ADCC and CDC mechanisms in addition to phagocytosis.

In specific embodiments, CD47×CD20 bispecifics provided herein, designated TPP-1360, TPP-1361, and TPP-1367 comprise heavy and light chain sequences as follows: TPP-1360 comprises (CD47 LC SEQ ID NO:19; HC SEQ ID NO:20)×(CD20 LC SEQ ID NO:15; HC SEQ ID NO:16). TPP-1361 comprises (CD47 LC SEQ ID NO:17; HC SEQ ID NO:18)×(CD20 LC SEQ ID NO:15; HC SEQ ID NO:16). TPP-1367 comprises (CD47 LC SEQ ID NO:21; HC SEQ ID NO:22)×(CD20 LC SEQ ID NO:15; HC SEQ ID NO:16).

These bispecific antibodies exhibit high affinity to CD20 and detuned affinity to CD47, showing effective CD47 blocking, cyno-cross reactivity, good physicochemical properties (solubility, stability, expression), and low immunogenicity prediction (EpiVax). See Example 15. The IgG1 heterodimer format and Fc confer reliable production in sufficient amounts and purity using standard CHO processes, with phase appropriate titer, yield, product quality and liquid formulation. These species exhibit in vitro phagocytosis capacity of CD20⁺ tumor cells superior to CC-90002 and more potent ADCC than rituximab. These species also exhibit marked reduction in cyno B cells in peripheral blood and lymphoid tissues. These highly evaluated species also exhibit minimal sink effects with no binding to CD20⁻CD47⁺ healthy cells (RBC and platelets). Importantly, the species exhibit acceptable PK parameters to support dosing described herein.

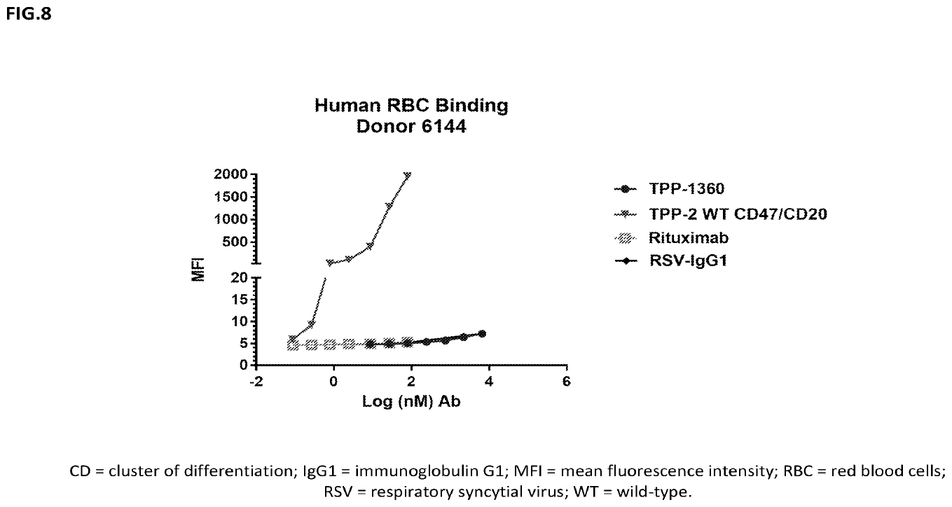

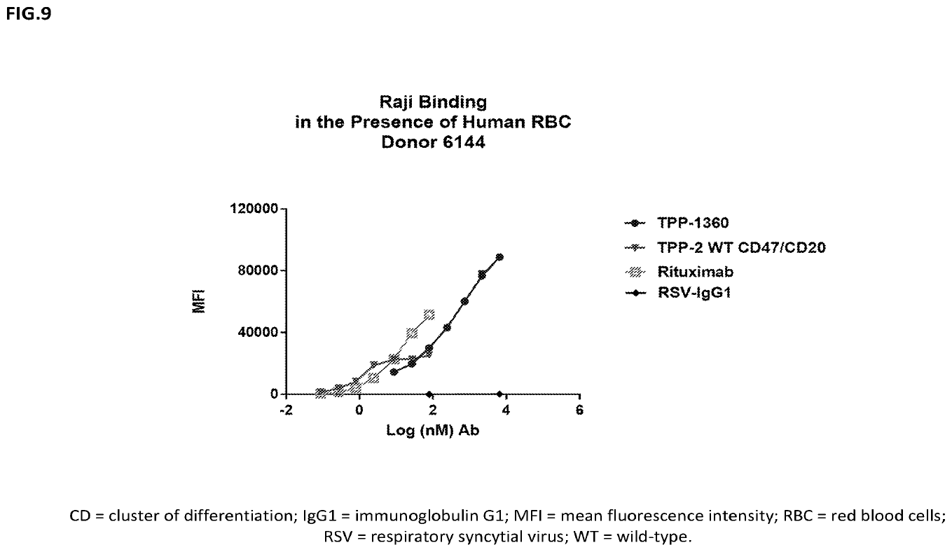

RBC binding capacity of TPP-1360, for example, was extensively evaluated in purified human RBCs and in co-culture of human RBCs with tumor cells. As illustrated in FIG. 8, TPP-1360 is demonstrated to selectively bind CD47⁺/CD20⁺ Raji Cells but Not CD47⁺/CD20⁻ human RBCs. Moreover, in a co-culture of Raji cells and human RBCs, TPP-1360 displayed dose-dependent binding to CD47⁺/CD20⁺ Raji cells but no binding to human RBCs, even at concentration as high as 1 mg/mL. See, FIG. 9. To the contrary, the CD47 wild type/CD20 bispecific, TPP-2, significantly binds both Raji cells and human RBCs. In addition, TPP-1360 does not show binding to purified cyno RBC from multiple donors.

The TPP-1360 species bispecific entity of the present disclosure, for example, is a first-in-class antibody, co-targeting CD47 and CD20, designed to bind CD20 with high affinity and CD47 with optimally detuned affinity. When bound to CD20 expressing cells, TPP-1360, for example, not only blocks macrophage checkpoint inhibitor SIRPα interaction with CD47 but also engages activating FcγRs to fully potentiate macrophages to engulf and destroy CD20 positive cells. Potent in vitro activity is induced by TPP-1360, for example, to eliminate tumor cells associated with lymphoid malignant neoplasm conditions described herein via multiple modes of action, including phagocytosis, ADCC and CDC. TPP-1360, exemplary of the bispecific entities described herein, provides enhanced pharmacological activities over rituximab and CC-90002.

TPP-1360, an IgG1 bispecific molecule, is designed to bind CD20 with high affinity and CD47 with optimally detuned affinity. TPP-1360 demonstrates selective binding to CD20⁺CD47⁺ cells. When bound to CD20 and CD47 expressing cells, TPP-1360 not only blocks macrophage checkpoint inhibitor SIRPα interaction with CD47 but also engages in activating FcγRs through the WT IgG1 to fully potentiate macrophage effector function to engulf and destroy CD20 positive cells. Overall, potent in vitro activity is induced by TPP-1360 to eliminate cancer cells via multiple modes of action, including phagocytosis, ADCC and complement dependent cytotoxicity (CDC) (FIG. 22).

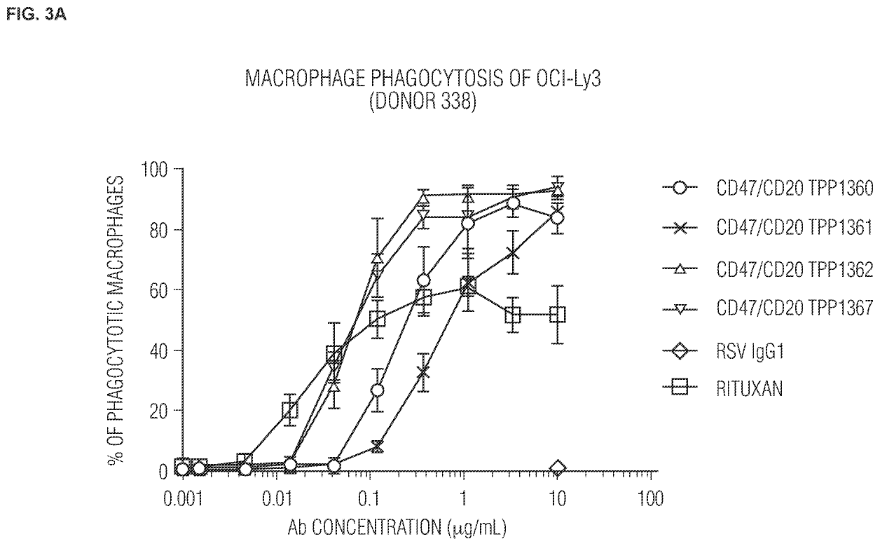

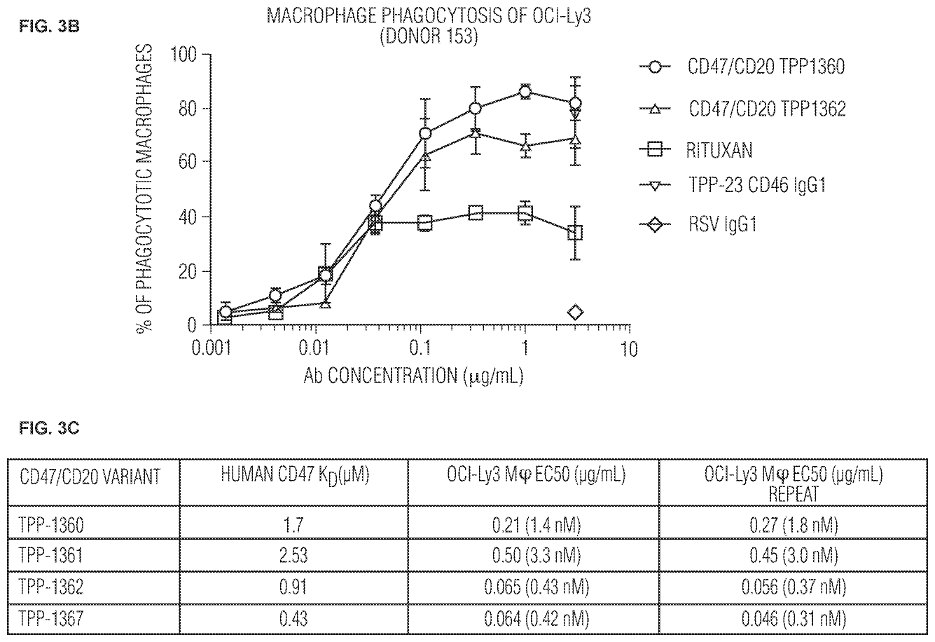

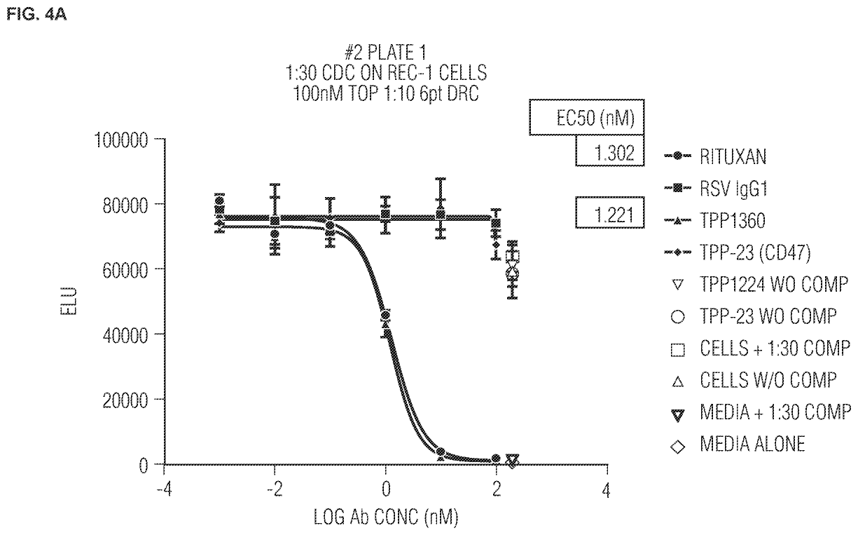

TPP-1361 is also an IgG1 bispecific antibody, co-targeting CD47 and CD20, designed to bind CD20 with high affinity and CD47 with optimally detuned affinity. Furthermore, TPP-1367 is also an IgG1 bispecific antibody, co-targeting CD47 and CD20, designed to bind CD20 with high affinity and CD47 with optimally detuned affinity. See FIGS. 3A-3C.

Once bound to CD20 on tumor cells the anti-CD47/anti-CD20 bispecific IgG1 heterodimeric antibody species described herein potently block CD47-SIRPα interaction and co-engage activating receptors FcγRs on effector cells through IgG1 Fc, resulting in activation of macrophage mediated phagocytosis and natural killer (NK) cell mediated cytotoxicity against tumor cells. The bispecific species described herein selectively bind CD47 on CD20 expressing tumor cells and are substantially free of binding to CD47 in normal cells. The ratio of binding to Raji (CD47+CD20+) vs human RBC in the co-culture binding assay for the bispecific species described herein is about 6,000 fold, for example. The ratio of binding to human B cells (CD47+CD20+) vs human RBC for the bispecific entities described herein is about 700 fold, for example. The level of selection of bispecific entities described herein exhibit selection in the range from about 400 to about 8,000 fold depending upon the expression level of CD20 and CD47 on tumor cells and normal cells. Accordingly, assuming a fixed level of CD47 expression, as CD20 levels increase bispecific entities described herein exhibit increased selectivity and potency.

In vitro affinity measurements with the extracellular domain of the effector antigen, CD47, initially revealed a 100-200-fold decrease in affinity for the bispecific IgG1 heterodimeric antibody species described herein. In vitro affinity measurements with the extra-cellular domain of CD47 revealed that the exemplified species had a 100-500-fold decrease in affinity. In vivo and in vitro cell based studies with these detuned IgG1 1+1 heterodimeric bispecifics confirmed effector-based cell killing and decreased binding to non-target cell types relative to a monospecific antibody.

Bispecific species described herein demonstrate selective binding to CD20-expressing cells, for example, wherein the interaction of CD47 with the macrophage checkpoint inhibitor, signal-regulatory protein alpha (SIRPα), is blocked. This increased selectivity over monospecific anti-CD47 approaches allows for the use of an IgG1 Fc, which engages activating fragment crystallizable gamma receptors (FcγRs) to fully potentiate macrophages to engulf and destroy CD20 positive cells. In comparison to the anti-CD20 antibody rituximab, for example, anti-CD47/anti-CD20 bispecific antibodies described and exemplified herein are more potent in inducing phagocytosis and ADCC.

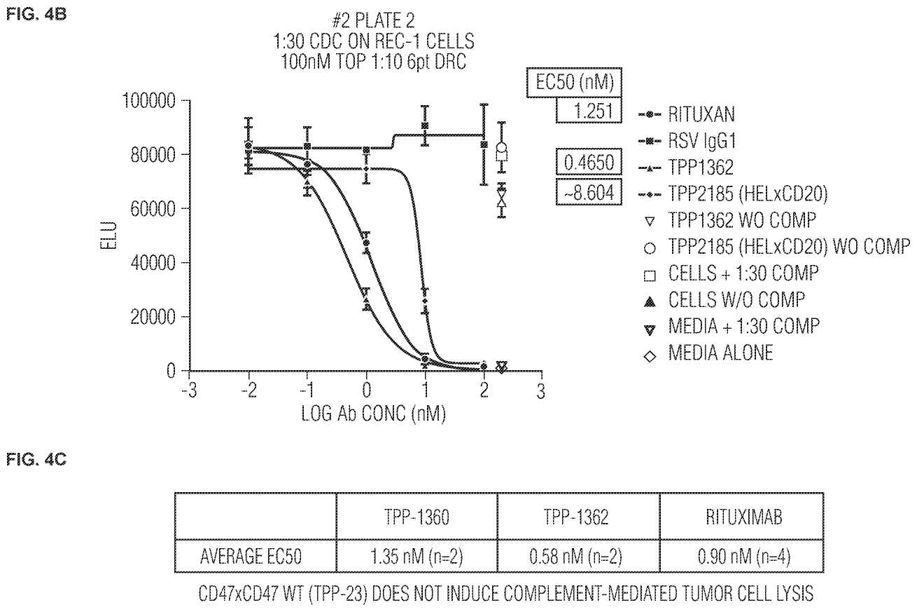

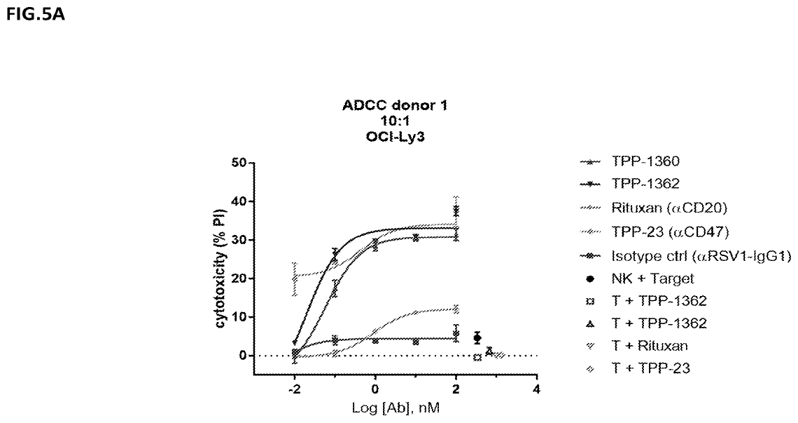

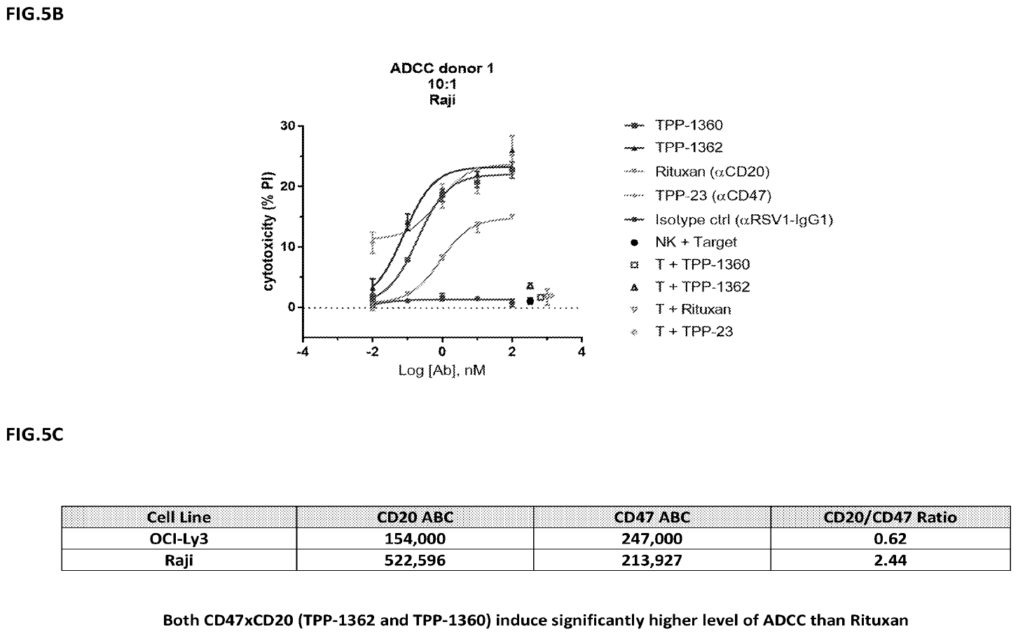

In vitro cell-based studies demonstrate that detuned CD47 bispecific entities described herein activate antibody-dependent cellular phagocytosis, complement-dependent cytotoxicity (CDC), and antibody-dependent cellular cytotoxicity (ADCC). See FIGS. 4A-4C and FIGS. 5A-5C. Further, cynomolgus (cyno) monkey pharmacokinetic (PK) and exploratory toxicity (E-tox) studies experiments demonstrate the detuned CD47 bispecifics effectively deplete B-cells and have reduced binding to cynomolgus red blood cells (RBCs) relative to the parental monospecific anti-CD47 antibody, thereby substantially confirming the success and medical value of the target-cell selective strategy described and claimed herein. Species exemplified herein demonstrate favorable pharmacokinetics and depletion of CD20⁺ B cells with minimum deleterious effects seen on hematologic parameters following multiple administrations to nonhuman primates.

Structural analysis demonstrated that TPP-1360 binds to the region of CD47 previously identified to be recognized by SIRPα. The interaction between recombinant SIRPα and human CD47 on cell surface is blocked with a TPP-1360 50% inhibitory concentration (IC50) value in the nanomolar range. See Example 6. TPP-1360 was evaluated as single agent in co-culture assays with tumor cells and human monocyte derived macrophages. TPP-1360 enabled antibody-mediated phagocytosis of a panel of NHL cell lines in vitro. See Examples 6 and 12. The maximum phagocytosis index for this panel ranged from approximately 28% to 86% for all the cell lines tested. Antibody concentration-response studies indicated that the TPP-1360 effect was concentration-dependent in the lymphoma lines with EC50 values in the subnanomolar range.

Characterization of the Fc portion of TPP-1360 included Fc gamma receptor (FcγR) binding, complement-dependent cytotoxicity (CDC), antibody-dependent cellular cytotoxicity (ADCC), and cytokine release assays (CRA). TPP-1360 binds to FcγRs with low to high affinity depending on the subtype. TPP-1360 exhibits ADCC in a cellular co-culture assay employing human NK cells and a panel of NHL cell lines. Additionally, CDC was observed with CD47+CD20+ DLBCL as the target cells. TPP-1360, at concentrations up to 200 nM, induced minimal cytokine release from multiple human donor PBMCs in plate bound format, similar to rituximab or isotype control antibody.

TPP-1360 demonstrated a high level of binding to human and cynomolgus macaque B cells, and a low level of binding to NK cells. See Example 11. In both human whole blood and peripheral blood mononuclear cells (PBMC), the major immune subset recognized by TPP-1360 was CD19+CD14-B cells. In human whole blood, TPP-1360 was able to deplete B cells in a concentration dependent manner with IC50 values in the subnanomolar. TPP-1360, at concentrations up to 1333.3 nM, did not bind to red blood cells (RBCs) from human or cynomolgus monkeys. Consistent with its reduced affinity for human CD47 and no binding to RBCs, TPP-1360 did not induce hemagglutination of human erythrocytes at concentrations up to 1333.3 nM.

Studies were conducted using surface plasmon resonance to evaluate the binding of TPP-1360 to CD47. See Example 4. TPP-1360 demonstrated recent calculated example data equilibrium constants (K_{D}) values of 2.32±0.11 μM for the extracellular domain of human CD47, 23 μM ±0.14 for cynomolgus CD47, and no binding to mouse CD47. The detuned anti-CD47 epitope was determined by crystallography. The VH CDRs make multiple contacts to the KGRD loop of CD47 and the VL CDRs overlap with the SIRPα binding site, which explains the ability to block SIRPα binding.

Wild-type (WT) IgG1 sequence employed in its CH2 domain, TPP-1360 demonstrated calculated K_{D} values ranging from 0.04 nM to 1624 nM for the recombinant human fragment crystallizable gamma receptor (FcγR) family members tested. Compared to the control anti-respiratory syncytial virus (RSV) IgG1 antibody, TPP-1360 showed higher affinity to FcγR2A (H131), FcγR2A (R131), FcγR3A (F176), and FcγR3A (V176) recombinant proteins with p value of 0.0038, 0.00019, 0.0045, and 0.0049, respectively. Additionally, the ability of TPP-1360 to bind to different FcγR-engineered HEK293 cells was assessed by time-resolved fluorescence resonance energy transfer (TR-FRET). Although no difference in TPP-1360 binding to the FcγR1 cell line was observed when compared to the control anti RSV IgG1 antibody, TPP-1360 demonstrated higher binding to the FcγR2A, FcγR2B, and FcγR3A cell lines when compared to the control anti RSV IgG1 antibody, indicating higher affinity to bind natural killer (NK) cells and enhanced capacity to activate NK-mediated antibody dependent cellular cytotoxicity (ADCC).

TPP-1360 as a single agent was compared to the combination of rituximab and TPP-356, a CD47 mAb with non-attenuated affinity to CD47 and an immunoglobulin G4 with serine 228 to proline and leucine 235 to glutamic acid mutations (IgG4PE), that has a detuned fragment crystallizable (Fc). TPP-1360 single-agent activity in macrophages was higher than either rituximab or TPP-356 alone and was equivalent to the combination of TPP-356 and rituximab in inducing macrophage-mediated phagocytosis as shown in one representative graph (FIG. 23).

The detuned CD47 arm contributes to cellular functions, as evidenced by, for example:

a) CD47×CD20 bispecific demonstrated enhanced phagocytosis compared to rituximab or CC-90002 as a single agent. Phagocytic activity of CD47×CD20 bispecific in general correlates with their CD47 binding affinity. In addition, TPP-1360 single agent activity is equivalent to the combination of CC-90002-like anti-CD47 IgG4PE antibody (TPP-356) and rituximab in inducing phagocytosis.

b) TPP-1360 demonstrated better ADCC than rituximab in rituximab-sensitive and resistant tumor cells.

c) TPP-1360 also demonstrated better efficacy than rituximab in vivo in Raji NOD-SCID xenograft model.

TPP-1360 differentiates from T cell engager bispecific antibodies targeting CD20 and CD3 (CD20×CD3) currently being tested in clinical trials. As TPP-1360 has different modes of action that include phagocytosis, ADCC, and CDC, versus T cell activation, TPP-1360 exhibits less risk of Cytokine Release Syndrome (CRS) compared with CD20×CD3 bispecific antibodies. Some of these trials such as NCT02500407 (mosunetuzumab), NCT03075696 (glofitamab) and NCT02290951 (REGN1979) reported CRS rates of 28.4%, 57.1% and 57.3%, respectively (Schuster S J, et al. Mosunetuzumab induces complete remissions in poor prognosis non-Hodgkin lymphoma patients, including those who are resistant to or relapsing after chimeric antigen receptor T-cell (CAR-T) therapies, and is active in treatment through multiple lines. Blood. 2019; 134(S1):6; Morschhauser F, et al. Dual CD20-Targeted Therapy With Concurrent CD20-TCB and Obinutuzumab Shows Highly Promising Clinical Activity and Manageable Safety in Relapsed or Refractory B-Cell Non-Hodgkin Lymphoma: Preliminary Results From a Phase Ib Trial. Blood 2019; 134(S1):1584; Bannerji R, et al. Clinical Activity of REGN1979, a Bispecific Human, Anti-CD20×Anti-CD3 Antibody, in Patients with Relapsed/Refractory (R/R) B-Cell Non-Hodgkin Lymphoma (B-NHL) Blood 2019:134(S1):762). Furthermore, TPP-1360 demonstrates favorable pharmacokinetics with minimal deleterious effects seen on hematologic parameters, namely RBC, following multiple administrations to nonhuman primates. TPP-1360 is particularly developed as an intravenous (IV) injectable treatment for CD20+ B-cell lymphoma patients refractory and/or resistant to current therapies.

### In Vivo Pharmacology

The in vivo efficacy of TPP-1360 was evaluated in two lymphoma cell line-derived xenograft models. Significant dose-dependent antitumor activity was observed with TPP-1360 treatment in the WSU-DLCL2, a DLBCL cell line xenograft model. Significant, but not dose-independent, tumor inhibition was achieved with TPP-1360 in Raji, a Burkitt lymphoma cell line xenograft model.

Overall, TPP-1360 exhibits an acceptable safety profile for the intended patient population, and the toxicology program adequately supports the conduct of clinical trials in patients with advanced cancer. See Example 1.

TPP-1360 was tolerated in monkeys in all studies, including the repeat-dose GLP toxicology study up to and including the highest dose evaluated (100 mg/kg administered weekly for 5 doses).

The safety profile of CD20 targeting is well established, and TPP-1360 effects in cynomolgus monkeys were consistent with expected pharmacology. The TPP-1360 anti-CD20 arm derived from rituximab is paired with a detuned anti-CD47 arm that relies on CD20 engagement for binding to target. The overall binding profile of TPP-1360 in human whole blood is similar to rituximab. See Example 11. Rituximab is widely used for NHL at an approved dose of 375 mg/m² (approximately 10 mg/kg for a 70 kg subject) weekly that is well-tolerated.

The detuned anti-CD47 arm of TPP-1360 (Hu CD47 K_{D} 2.32±0.11 μM) is derived from anti-CD47 monoclonal antibody CC-90002 (Hu CD47 K_{D} 0.54±0.37 nM). CC-90002 was well-tolerated up to 20 mg/kg in combination with rituximab (375 mg/m²) in subjects with R/R NHL (Abrisqueta P, et al. Anti-CD47 Antibody, CC-90002, in Combination with Rituximab in Subjects with Relapsed and/or Refractory Non-Hodgkin Lymphoma (R/R NHL). Blood. 2019; 134 (Supplement_1): 4089).

Many CD47 antibodies have been reported to cause hemagglutination of human erythrocytes, and this represents a major limitation of therapeutically targeting CD47 with existing IgG antibodies that retain Fc function due to hemolytic anemia. TPP-1360 did not promote hemagglutination in human RBC at concentrations of up to 1333.3 nM (200 μg/mL), consistent with its reduced affinity to human CD47 and lack of binding to human RBCs. In addition, no RBC binding was observed with TPP-1360 using an antiglobulin (Coombs) test at concentrations of up to 300 μg/mL. There were only minimal decreases in red blood cells in cynomolgus monkeys at 100 mg/kg Q1W (Cₘₐₓ of 4640 μg/mL, 58.7-fold greater than the projected Cₘₐₓ at the example clinical start dose).

TPP-1360 induced minimal levels of cytokine release in a CRA using plate-bound TPP-1360 at concentrations of up to 200 nM (30 μg/mL), similar to rituximab, indicating low risk of cytokine release in humans.

Safety data is well established for rituximab and clinical experience with the parent anti-CD47 antibody. CC-90002 (20 mg/kg), in combination with rituximab at 375 mg/m² (approximately 10 mg/kg for a 70 kg subject) demonstrated the combination was well tolerated.

### Conditions

Efficacy, Safety, and tolerability of TPP-1360 and related entities described herein is provided for the treatment of lymphoid malignant neoplasm conditions, including NHL, for example, particularly in subjects with relapsed or refractory CD20+ NHL who have progressed on standard anticancer therapy or for whom no other approved conventional therapy exists. Non-Hodgkin's lymphoma is expected to express CD20 antigen such as diffuse large B-cell lymphoma (DLBCL), Grade 1, 2, 3a and 3b follicular lymphoma (FL), marginal zone lymphoma (MZL), and mantle cell lymphoma (MCL).

Efficacy, Safety, and tolerability of TPP-1360 and related entities is provided for the treatment of diffuse large B-cell lymphoma (DLBCL), for example, particularly in subjects with relapsed or refractory DLBCL who have progressed on standard anticancer therapy or for whom no other approved conventional therapy exists. Diffuse large B cell lymphoma (DLBCL), not otherwise specified (NOS; includes transformed DLBCL from indolent histology, high grade B-cell lymphoma with DLBCL histology, primary mediastinal B-cell lymphoma, and follicular lymphoma Grade 3b).

Efficacy, Safety, and tolerability of TPP-1360 and related entities is provided for the treatment of follicular lymphoma (FL), for example, particularly in subjects with relapsed or refractory FL who have progressed on standard anticancer therapy or for whom no other approved conventional therapy exists.

NHL subjects, for example, may be required to have bi-dimensionally measurable disease (at least one nodal lesion >1.5 cm in its longest diameter or at least one extranodal lesion >1.0 cm in its longer diameter) on cross sectional imaging by CT or MRI as defined by Lugano criteria (Cheson B D, et al. Recommendations for initial evaluation, staging, and response assessment of Hodgkin and non-Hodgkin lymphoma: the Lugano classification. J Clin Oncol. 2014; 32(27):3059-3068).

Subjects for treatment described herein may be required to exhibit one or more of the following:
Absolute neutrophil count (ANC) ≥1.0×10⁹/L without growth factor support for 7 days (14 days if on pegfilgrastim).
Hemoglobin (Hgb) ≥8 g/dL without transfusion for 14 days.
Platelets (plt) ≥75×10⁹/L without transfusion for 7 days.
Aspartate aminotransferase (AST/SGOT) and alanine aminotransferase (ALT/SGPT) ≤2.5×Upper Limit of Normal (ULN) or ≤5.0×ULN if tumor is present in the liver.
Serum bilirubin ≤1.5×ULN.
Estimated serum creatinine clearance of ≥45 mL/min using the Cockcroft-Gault equation or measured creatinine clearance using 24-hour urine collection.

International normalized ratio (INR) <1.5×ULN and partial thromboplastin time (PTT) <1.5×ULN.

The presence of any of the following may exclude a subject from treatment described herein:
Burkitt's or lymphoblastic lymphoma.
Chronic lymphocytic leukemia (CLL) or small lymphocytic lymphoma (SLL) including Richter's transformation.

### IV Administration

TPP-1360, for example, solution for injection is provided as liquid in vials at a concentration of 50 mg/mL packaged in cartons and labeled appropriately in accordance with United States Food and Drug Administration (FDA) requirements and Good Clinical practice (GCP) standards. The solution for injection drug product is stored at 2° to 8° C.

TPP-1360 dosage and regimen will be based on the totality of available data that include nonclinical toxicology, in vitro studies, and clinical information.

A manufacturing process for bispecific antibodies described herein may follow a typical Chinese Hamster Ovary (CHO) manufacturing platform. A common contaminant observed in the purification of these bispecific antibodies is the half-antibody, which requires specific purification protocols to remove. After expression of the 4 chain bispecific in Chinese Hamster Ovary cell, protein A is used as the first step to purify an IgG based bispecific. Following this first step there are generally two species present, the desired 4 chain bispecific and a half-antibody. In most cases ion exchange chromatography is sufficient to separate these two species, but in others hydrophobic interaction chromatography may be required. Correct pairing of the LCs should be assessed by mass spectrometry and misassembled impurities should be removed by additional protein purification methods, such as ion exchange or hydrophobic interaction chromatography. Following either secondary purification approach, preparative size exclusion chromatography (SEC) can be used to polish and ensure conformational homogeneity, while buffer exchanging the 4 chain bispecifics. Final quality control should include analytical SEC, mass spectrometry, and in vitro binding assessments with the different antigens to ensure the conformational and chemical integrity of the bispecific. See, e.g., J. B. Ridgway et al., Protein Eng. 9 (1996) 617-621. K. Gunasekaran et al., J. Biol. Chem. 285 (2010) 19637-19646.

### EXAMPLES

### Example 1: Toxicology

The cynomolgus monkey was selected as the single relevant species for nonclinical toxicity assessment based on species homology, antibody binding affinity to CD47 and CD20 across species, and functional assessment in cynomolgus monkey. Cynomolgus monkey CD47 and CD20 are highly homologous to human CD47 and CD20, while rat and mouse CD47 and CD20 have less homology. Further, TPP-1360 binds to human and cynomolgus monkey CD47 and CD20 with similar affinity, and does not bind to mouse CD47 or CD20. TPP-1360 causes marked depletion of peripheral blood B cells in cynomolgus monkey, demonstrating functional activity in vivo. Based on this information, cynomolgus monkey was selected as the single relevant toxicology species.

Two general toxicity studies were conducted in cynomolgus monkeys to characterize the potential toxicity of TPP-1360: a 2-week exploratory study with a 14-day non-dosing period, and a one-month GLP-compliant toxicity study. The studies were conducted via IV injection.

Effects in both monkey toxicology studies were generally similar. Administration of TPP-1360 to cynomolgus monkeys, either BIW or Q1W, resulted in the expected marked decreases in B-cells in peripheral blood and decreased lymphoid cellularity primarily in the follicular regions of the lymphoid organs, consistent with the pharmacology and observed with other anti-CD20 agents such as Rituxan and Gazyva (Rituxan® [Product Monograph]. Mississauga, ON, Canada: Hoffmann-La Roche Ltd.; 2000, revised 2019; Gazyva FDA Pharmacology Review 125486, 2013). Other hematologic effects included moderate to marked decreases in NK cells (reported clinically for Rituxan; Enqvist M, et al. Systemic and Intra-Nodal Activation of NK Cells After Rituximab Monotherapy for Follicular Lymphoma. Front Immunol. 2019. 10:2085; transient decreases reported for Gazyva in cynomolgus monkeys), and minor variable non-dose-dependent decreases in T cell populations were also noted (CD8 and CD4 T cells) and for the latter, a relationship to treatment could not be ruled out. A range of other adverse and non-adverse changes with no dose response were also noted that reflect an immune/inflammatory response secondary to TPP-1360 administration to cynomolgus monkeys, including ADA generation and immune complex deposition.

TPP-1360 administration in cynomolgus monkeys resulted in expected decreases in B cells in peripheral blood and decreased cellularity of lymphoid organs, NK cells, and red blood cells, as well as other effects considered secondary to administration of a humanized protein to cynomologus monkeys. B cell effects of rituximab and its biosimilar in peripheral blood and lymphoid tissues have also been shown to be reversible in cynomolgus monkeys (Rituxan® [Product Monograph]. Mississauga, ON, Canada: Hoffmann-La Roche Ltd.; 2000, revised 2019). Rituxan or Gavyza-mediated NK cell decreases are generally transient (Enqvist M, et al. Systemic and Intra-Nodal Activation of NK Cells After Rituximab Monotherapy for Follicular Lymphoma. Front Immunol. 2019. 10:2085; Gazyva FDA Pharmacology Review 125486, 2013), and CD47-mediated pharmacologic effects on red blood cells are also reversible. Furthermore, there was evidence of recovery of B cells, NK cells, and T cells in peripheral blood with decreasing serum TPP-1360 levels. In addition, there were no observable bone marrow effects, and reticulocytes were increased in some animals, indicating a regenerative response to the red cell decreases.

A No-Observable-Adverse-Effect-Level (NOAEL) was not determined in the one-month study, based on the B and NK cell effects at all dose levels.

### Example 2: Pharmacokinetics

Pharmacokinetics and toxicokinetics of TPP-1360 were evaluated in cynomolgus monkeys following a single IV dose and repeat IV dose studies. Using allometry-derived PK parameters, the projected human clearance for TPP-1360 is 25.6 mL/hour, and the predicted t % of TPP-1360 in humans is expected to be approximately 5 days.

### Example 3: Detuning of CC-90002

Rational design to decrease the affinity of the non-detuned parental version of CC-90002 (408_437) anti-CD47 arm was enabled with a crystal structure of an anti-CD47 Fab bound to the extra cellular domain of CD47. The epitope bound by CC-90002 is identical to that of original murine anti-CD47 2A1 bound to human CD47. See, U.S. Pat. No. 9,045,541.

The variable domains of 2A1 were humanized and the final antibody was named “ON” composed of HC_2.30 and LC_N, which ultimately was developed as an IgG4 PIE format (CC-90002). QN was further modified by the introduction of residues into the variable heavy domain for improved cell-free expression, this HC variant was named “HC_Q_5_MUT”. The HC_Q_5_MUT HC and LC_N were further modified to decrease their immunogenicity using in silico modeling and in silico prediction of immunogenicity, these were collectively referred to as “CD47 2.0”. Further variants in the variable heavy and variable light domains of CD47 2.0 LC_1147_2 and CD47 2.0 HC_434 were designed for improved pharmacokinetics, these were referred to as “CD47 3.0”. WO2016109415 (US.20170369572); WO2018009499 (US.20190241654); and WO2018183182, each of which are herein incorporated by reference.

The anti-CD47 epitope covers a large surface area and residues from both the light chain (LC) and the heavy chain (HC) participate in the interaction.

To decrease the affinity of the anti-CD47 arm for CD47, CD47 interacting residues from both the LC and HC were subjected to in silico mutagenesis using the “Residue Scan” module from the Molecular Operating Environment (MOE) modeling program. This process created a library of thousands of variants with a wide range of predicted affinities. Each in silico Fab variant was modeled to calculate a predicted change in stability (dStability) or a change in affinity for the CD47 ECD (dAffinity). Over 5,000 variants with positive dAffinity scores (predicted to have lower affinity relative to the parental Fab) and negative dStability (predicted to have higher stability than the parental Fab) were analyzed using immunogenicity assessment software to identify variants that would be predicted to have low immunogenicity. Of these, 143 low immunogenic risk Fab variants with predicted Kds for CD47 ranging from 10 nM to 1 mM, were selected for cell based testing.

To screen target-cell selective anti-CD47 Fabs, the selected anti-CD47 Fab variants were constructed as IgG1 fusions and paired with the anti-EGFR arm from cetuximab. The proper assembly of the 4 chain bispecific was enabled by the presence of Fab and Fc substitutions described herein present in all 4 chains. The 4 chain bispecifics containing the 143 selected variants were transiently expressed in Expi-CHO cells and the bispecifics were purified in a single step using magnetic protein A beads. To identify the target-cell selective bispecifics, the variants were tested with two experiments. The first experiment measured the ability of the detuned anti-CD47×anti-EGFR bispecifics to bind to the non-target Raji cell line that expressed the CD47 antigen, but not the EGFR antigen. The second experiment measured the ability of the detuned anti-CD47×anti-EGFR bispecifics to block SIRPα binding to the target Fadu cell line that expressed the CD47 antigen and the EGFR antigen. These experiments, yielded a set of 8 variants that showed a 10-fold to 20-fold decreased affinity for the non-target CD47+/EGFR-Raji cell line relative to the non-detuned anti-CD47×anti-EGFR parental antibody, and yet was still able to block 75-90% SIRPα binding to the CD47+/EGFR+ Fadu target cell line.

The rituximab anti-CD20 arm was paired with the 8 detuned anti-CD47 variants similarly using an IgG1 Fc. It was observed that the detuned CD47×CD20 bispecifics had reduced binding to the CD47⁺/CD20− non-target Fadu cell line relative to the non-detuned CD47×CD20 parental antibody, and yet were still able to block 75-90% of SIRPα binding to the target Raji cell line which was CD47 and CD20 positive.

Additional developability assessments of the variants led to the selection of a single anti-CD47 Fab variant, VH E59Y/S102E, which was cloned into three CC-90002 derived frameworks, for pharmacokinetic testing in cynomolgus monkeys: TPP-1367, TPP-1360, and TPP-1361.

### Example 4: Summary of SPR Binding Results for Bispecific Entities Described Herein

Surface Plasmon Resonance (SPR) experiments were used to measure the affinities of TPP-1360 to CD47. TPP-1360 was tested for binding to human CD47 and cynomolgus CD47, and were found to not bind to mouse CD47. TPP-1360 was measured to have an affinity for human CD47 ECD of 1.7 μM Kd, which reflects ˜350× decrease in affinity relative to the parental anti-CD47 binder. The TPP-1360 affinity for the cynomolgus CD47 ECD was found to be 4.51 μM Kd. in addition to the measured affinity, a sandwich SPR assay demonstrated that TPP-1360 bound CD47 and CD20 simultaneously.

### Example 5: Dose Response of Binding and SIRPα Blocking of Example Bispecific Entities

Dose response curves for TPP-1360 blocking of human SIRPα binding to various CD20 expressing non-Hodgkins lymphoma tumor cell lines were generated. Cell lines were incubated with increasing concentrations of the bispecific, then human SIRPα was added at a saturating concentration. In addition to the bispecific, rituximab and the parental anti-CD47 binder (TPP-23 which is 408_437 with IgG1) were included for reference. Cells were washed then incubated with a secondary antibody to measure the amount of SIRPα bound to the tumor cells. For cell line OCI-Ly3 (a DLBCL cell line), TPP-1360 was found to have an EC50=1.30 nM. For the Raji cell line (a B-lymphocyte Burkitt's lymphoma cell line) TPP-1360 was found to have an EC50=1.64 nM. The parental anti-CD47, TPP-23, was found to have an IC₅₀ of 0.11 nM for blocking human SIRPα binding to OCI-Ly3 cells as shown in FIG. 21. Rituximab had no effect on SIRPα binding.

### Example 6: Dose Response for Phagocytosis

Dose response curve for TPP-1360 activation of phagocytosis towards various CD20 expressing non-Hodgkins lymphoma tumor cell lines were generated. Human monocytes were differentiated into macrophages, which were then added to tumor cell lines that had been incubated with increasing concentrations of either bispecific. In addition to the bispecific entities, rituximab and the parental anti-CD47 binder (TPP-23) were included for reference. Fluorescence labeling of macrophages and tumor cells was used to measure the number of phagocytic events using an image based quantification method. For the OCI-Ly3 cell line, TPP-1360 was found to have an IC50=1.4 nM. For the Raji cell line, TPP-1360 was found to have an IC50=1.8 nM.

### Example 7: Binding Studies with Human and Cyno RBCs and Hemagluttination

Binding of certain bispecific entity examples to human and cynomolgus monkey RBCs was determined to assess their non-target cell binding potential. RBCs were isolated from whole blood and were incubated with increasing concentrations of the example bispecifics. Binding was expressed as a percentage of the amount of binding observed at 2 μg/ml of the parental anti-CD47 binder (TPP-23). At 200 μg/ml, TPP-1360 and TPP-1361 bound to <1% of that seen for the parental anti-CD47 binding to human RBCs. Similarly, at 200 μg/ml, TPP-1360 bound to <1% of that seen for the parental anti-CD47 binding to cynomolgus RBCs. Finally, the parental anti-CD47 binders for both leads demonstrated no hemagluttination of human RBCs at 200 μg/ml. Similarly, both TPP-1360 and TPP-1361 showed no hemagluttination at 200 μg/ml. BRIC6, a known hemagluttinating antibody was used as a positive control.

### Example 8: Binding Studies to Human PBMCs and Whole Blood

Binding of the bispecific entity species described herein to human Peripheral Blood Mononuclear Cells (PBMCs) was assessed. Relative to the parental anti-CD47 binder TPP-23 and rituximab, the TPP-1360 bispecific showed less binding to all cell types with the exception of the B-cells, which showed significant binding through the presence of the anti-CD20 Fab portion.

### Example 9: First Round Lead Cynomolgus PK

A cynomolgus PK experiment was carried out with example bispecific entity species described herein. B-cell depletion was observed. From these studies TPP-1360 was chosen for further study in a cynomolgus monkey exploratory toxicology (E-tox) study, as described in Example 10.

### Example 10: Second Round Lead Cynomolgus E-Tox

A cynomolgus E-tox experiment was carried out with TPP-1360. These studies showed that TPP-1360 was well tolerated, showed deep B-cell depletion, and achieved dose-proportional exposure, confirming the avoidance of spurious binding to normal CD47+ but CD20 negative cells. Thus, the bispecific antibody is target-cell selective.

### Example 11: In Vitro Pharmacology

### A. Human Whole Blood Binding

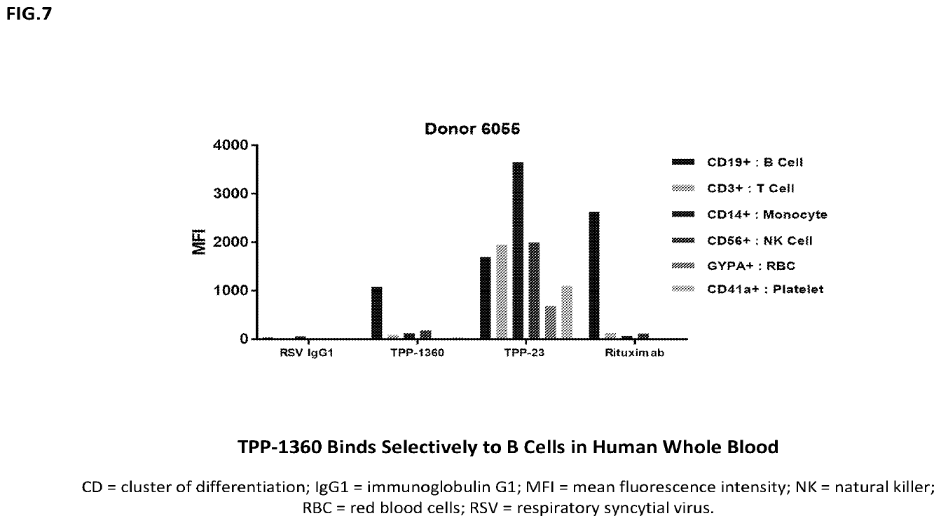

To assess the specificity of TPP-1360, its binding profile was first evaluated in whole blood using flow cytometry. Across two donors, 200 nM TPP-1360 substantially shifted the binding signal to B cells and rather weakly to T cells, monocytes, and NK cells, with minimal or no binding to platelets or red blood cells thereby illustrating selective binding to B cells in human whole blood. See, FIG. 7.

FIG. 7 shows that the bispecific TPP-1360, for example, binds primarily to B cells, with a very small amount of binding to the other cell types listed possibly because of higher levels of CD47 than what is found on blood cells, or because of the contribution of the Fc which can engage Fc receptors which are expressed on NK cells and monocytes. Conversely TPP-23, a high affinity CD47 monospecific antibody binds to all of these cell types due to the ubiquitous expression of CD47 and the high affinity for CD47 found in TPP-23.

The overall binding profile of TPP-1360 in human whole blood is similar to rituximab. Conversely, the parental CD47 mAb, TPP-23, used as a control for CD47 expression, significantly bound to all cell populations in human blood.

### B. Tumor Cell Binding

Furthermore, the RBC binding capacity of TPP-1360, for example, was extensively evaluated in purified human RBCs and in co-culture of human RBCs with tumor cells. As illustrated in FIG. 8, TPP-1360 selectively bound CD47⁺/CD20⁺ Raji Cells but not CD47⁺/CD20⁻ human RBCs. Moreover, in a co-culture of Raji cells and human RBCs, TPP-1360 displayed dose-dependent binding to CD47⁺/CD20⁺ Raji cells but no binding to human RBCs, even at concentration as high as 1 mg/mL. See, FIG. 9. To the contrary, the parental CD47 type/CD20 bispecific, TPP-2, significantly bound to both Raji cells and human RBCs. In addition, TPP-1360 does not show binding to purified cyno RBC from multiple donors.

### C. SIRPα Competition

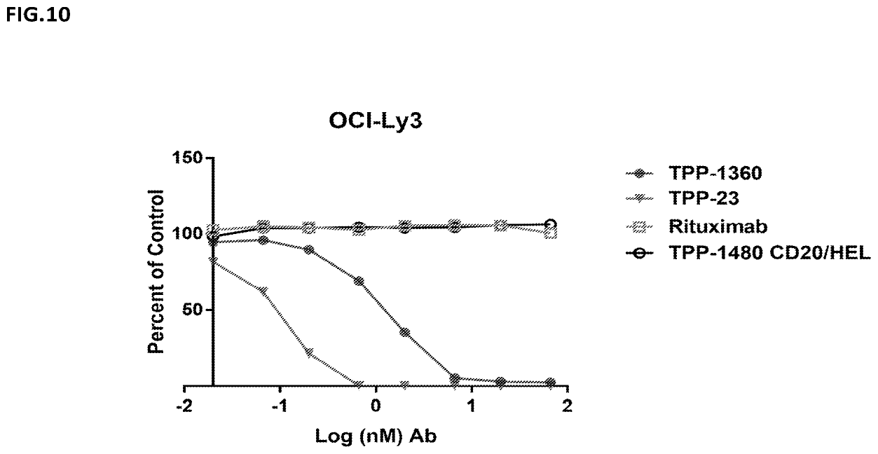

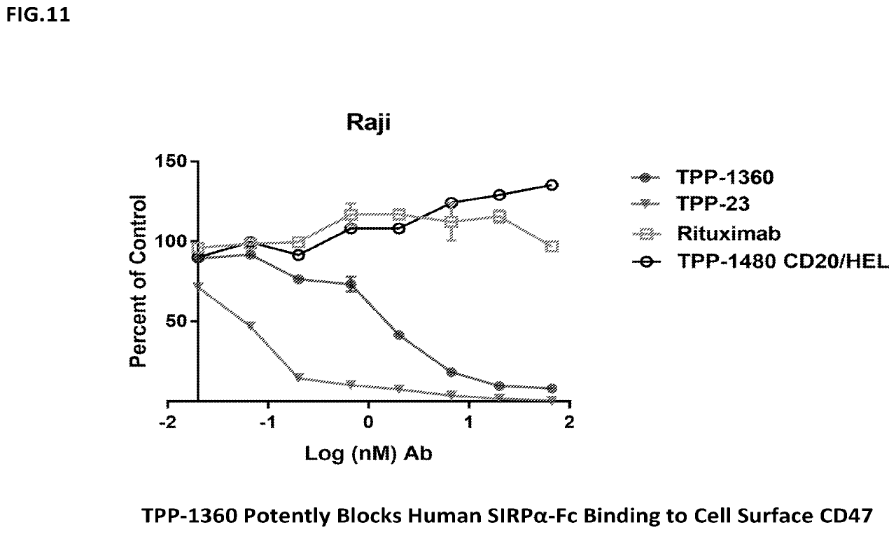

Having demonstrated its selective binding to CD20⁺/CD47⁺ cells, an assessment was made of the ability of TPP-1360 to antagonize human SIRPα interaction with cell surface CD47 using an in vitro competition assay. TPP-1360 potently and blocked recombinant human SIRPα-Fc binding to human CD47 expressed on the surface of CD20⁺/CD47⁺ lymphoma cell lines OCI-Ly3 and Raji, with average EC50 values of 1.30 nM and 1.64 nM, respectively. See, FIG. 10 and FIG. 11. FIG. 10 illustrates the fact that TPP-1360, for example, potently and completely blocked recombinant human SIRPα-Fc binding to human CD47 expressed on the surface of CD20⁺/CD47⁺ lymphoma cell line OCI-Ly3. FIG. 11 illustrates the fact that TPP-1360, for example, potently and completely blocked recombinant human SIRPα-Fc binding to human CD47 expressed on the surface of CD20⁺/CD47⁺ lymphoma cell line Raji. In contrast, neither rituximab nor control bispecific antibody TPP-1480 (anti-CD20/hen egg lysozyme) were able to compete with human SIRPα-Fc binding to the same cell lines. The data presented herein also demonstrates that TPP-1360 potency to block human SIRPα-CD47 interaction is lower than TPP-23, consistent with the attenuated affinity of TPP-1360 to human CD47.

### Example 12: Functional Activities: Human Macrophage Phagocytosis

This Example demonstrates the capacity of TPP-1360 in triggering tumor phagocytosis, as determined in vitro by automated counting of “eaten” CD20⁺ CD47⁺ tumor cells inside of labeled macrophages.

Expression of CD20 and CD47 was first verified in each target tumor cell line (OCI-Ly3, Raji, REC-1, and RIVA) by quantifying antibody binding capacity (ABC) using a flow cytometric assay (Denny T N et al., Cytometry. 1996 December; 26(4):265-74). All four cell lines express high levels of CD47 and CD20. Table 1.

TABLE 1
CD47 and CD20 Antigen Expression on Lymphoma Cell Surface

| Cell Line | CD20 ABC | CD47 ABC |
|---|---|---|
| OCI-Ly3 | 154,000 | 247,000 |
| REC-1 | 510,036 | 453,415 |
| RIVA | 722,000 | 443,000 |
| Raji | 522,596 | 213,927 |

ABC = Antibody binding capacity.

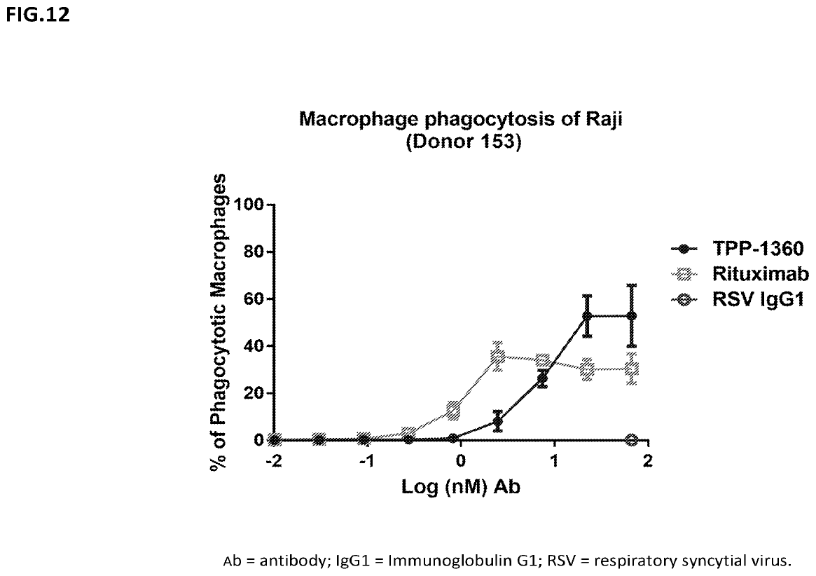

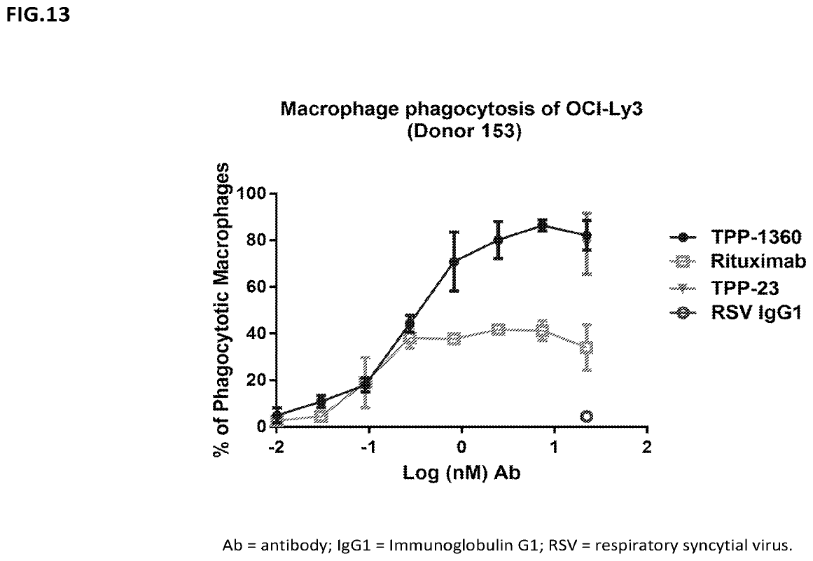

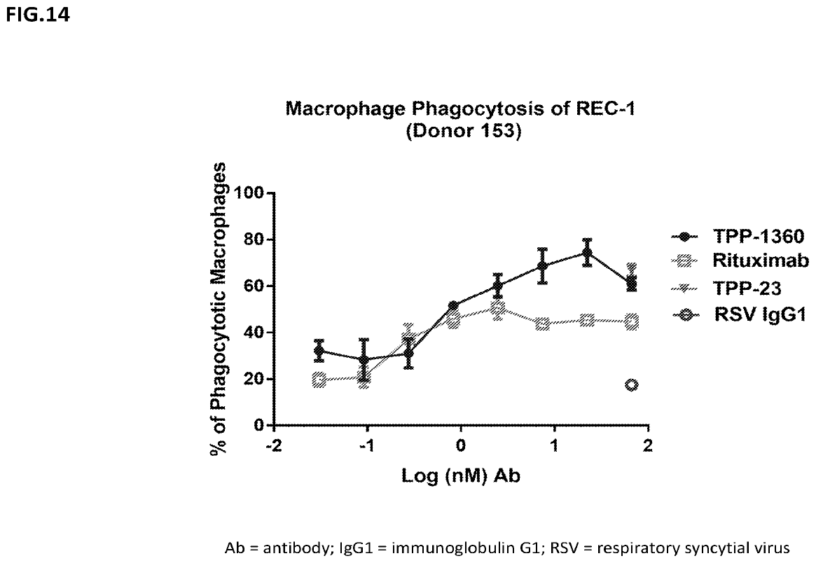

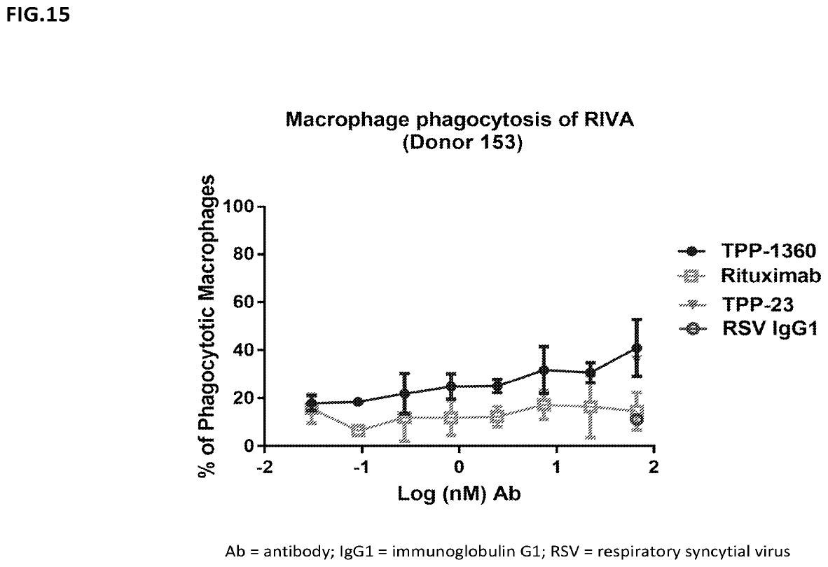

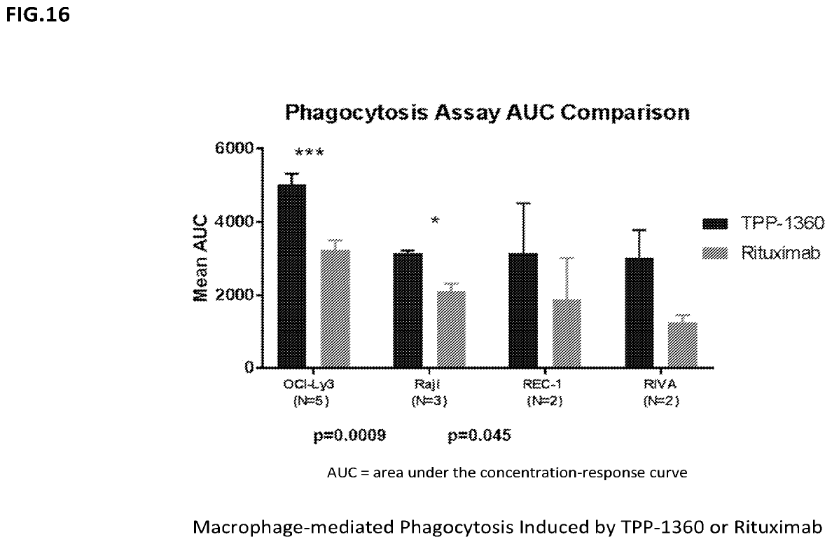

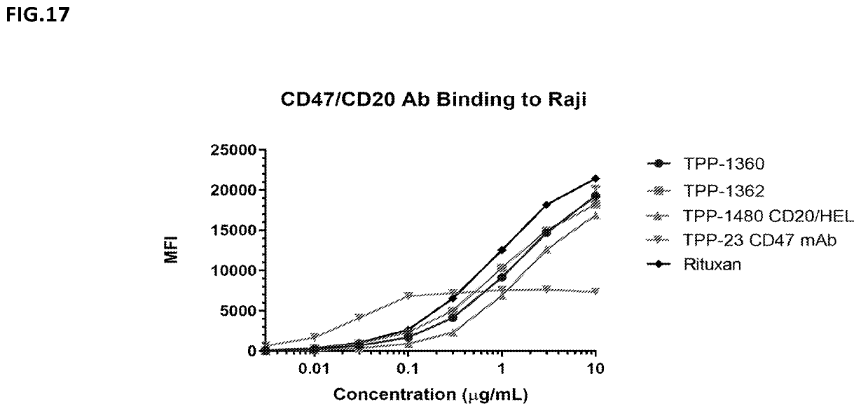

Next, titrated antibodies were added to pre-differentiated macrophages, followed by co-culture with carboxyfluorescein succinimidyl ester (CSFE)-labeled tumor cells opsonized with TPP-1360. Phagocytosis activity was quantitatively determined by the number of labeled tumor cells within the labeled macrophages. Green intensity (CFSE) was measured in each of the CD14 allophycocyanin (APC)-labeled macrophages, and a threshold gate was used to identify CFSE-positive macrophages. A threshold of approximately 1000 MFI (mean fluorescence intensity), with a variance of no more than a few hundred MFI was observed across the experiments. For each sample, the calculated percentage of phagocytosis was determined as: [(Number of CFSE-positive macrophages)/(number of total macrophage)]×100. Across at least two donors, treatment with TPP-1360 induced macrophage-mediated phagocytosis of four CD20⁺ malignant B cell lines. Representative data from one donor is shown in FIG. 12 (Raji cells), FIG. 13 (OCI-Ly3 cells), FIG. 14 (REC-1 cells), and FIG. 15 (RIVA cells). Area under the curve was calculated and followed by paired t test to determine statistical significance of TPP-1360 compared with rituximab. See, FIG. 16. The data demonstrates that treatment with TPP-1360 triggered significantly more efficient phagocytosis than rituximab in Raji and OCI-Ly3 cells, likely due to the concomitant blockade of the SIRPα-CD47 interaction and the engagement of activating receptors, such as FcγRs, by TPP-1360.

### Example 13: Pharmacokinetics

To determine the pharmacokinetic (PK) profile of the bispecific entities (antibody species, TPP-1360, TPP-1361, and TPP-1367 described herein, non-GLP studies were conducted in mice and cynomolgus monkeys. Sparse PK sampling (n=4 per timepoint) was performed over the course of 72 hours, and all animals showed detectable antibody species concentrations throughout study duration. The calculated half-life was 3.4 days but may be underestimated considering the sampling duration. To evaluate the PK profiles in cynomolgus monkeys, a repeat-dose exploratory toxicology study was performed. Following repeat-dosing of the antibody species, systemic exposure was achieved, and the antibody species was detectable in the serum of 2 of 3 monkeys throughout study duration (336 hr post Day 15 dose). Samples were also collected as part of the repeat-dose study for hematology and immunophenotyping assessments. The observed depletion of B-lymphocytes demonstrates drug functionality in vivo. Overall, antibody species exposure was maintained throughout study duration in both the single-dose mouse and repeat-dose monkey studies with similar half-lives ranging from 3-3.5 days reported between the two studies.

### Example 14: Safety Profile

These highly evaluated species exhibit acceptable toxicology profile. Toxicokinetics were evaluated as part of a 28-day exploratory toxicology study in cynomolgus monkeys. Serum concentrations were measured with a sandwich ELISA using an anti-rituximab antibody for capture and a goat anti-human IgG Fc for detection. Following multiple IV doses systemic exposure of TPP-1360 was achieved at all dose levels and maintained by all animals throughout study duration. TPP-1360 exhibited linear TK with approximately dose proportional increases in Cₘₐₓ and AUC₀₋₁₆₈. The mean calculated half-life ranged from 2-4 days depending on the dose level and dose regimen. Anti-drug antibody was detected in 5/8 animals tested at Day 15 prior to dose and in 5/6 animals tested on study Day 29. Anti-drug antibodies did affect the exposure of TPP-1360 as evidenced by an observed decrease in exposure for ADA positive animals. However, no test article-related decrease of platelets was observed. Decreases in T cells, NK cells, neutrophils and red blood cells are believed to be mediated by the CD47 arm of TPP-1360, since these cells do not express CD20.

### Example 15: Immunogenicity

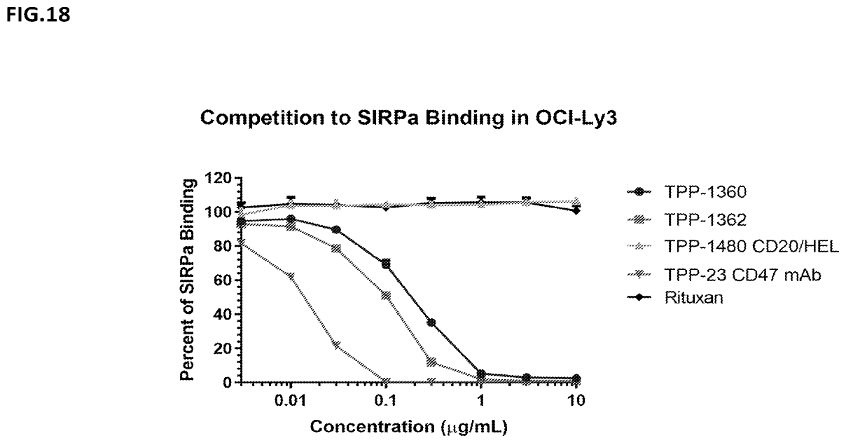

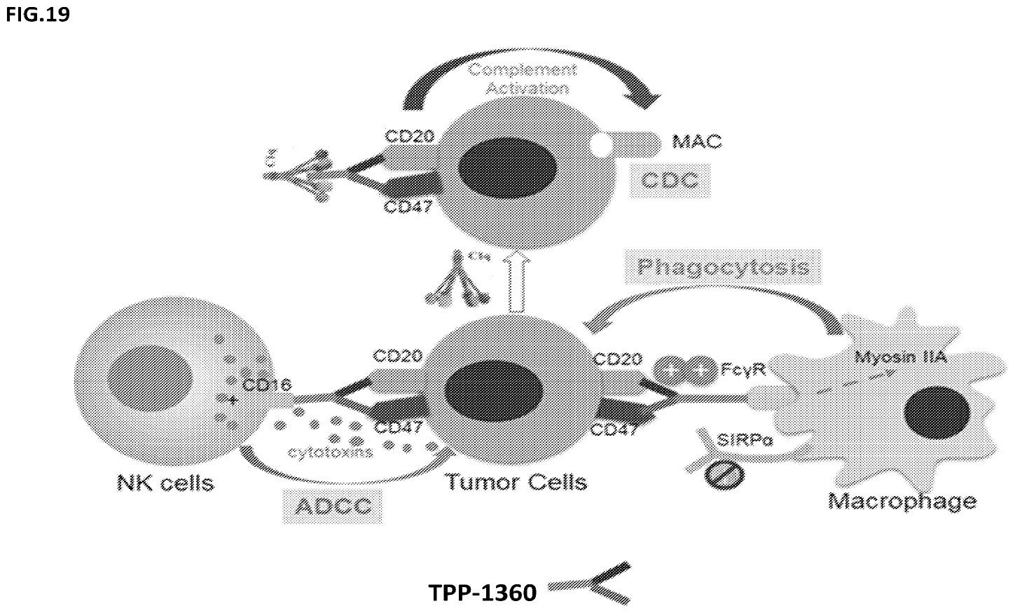

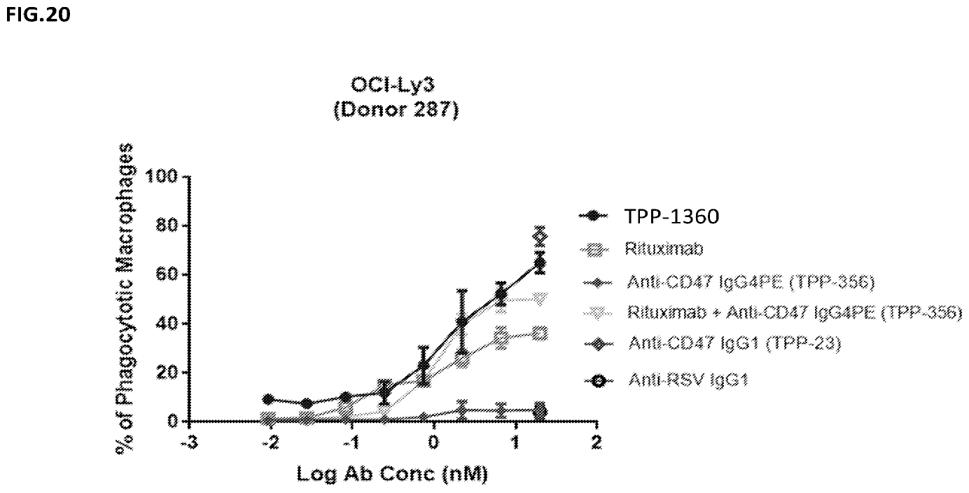

The Interactive Screening and Protein Reengineering Interface (ISPRI) software, developed by EpiVax, is an in silico computational method used to assess potential antibody immunogenicity in humans, and is known to be a clinically well-established T cell-dependent analysis tool (FIG. 18). The VH and VL amino acid sequences of TPP-1360 were analyzed for putative T effector and T regulatory hotspots and were found to have a low risk for immunogenicity.

### Example 16: Clinical Study: Design and Inclusion and Exclusion Criteria

A first-in-human clinical study of a CD47×CD20 bispecific antibody (“the bispecific” or “the bispecific antibody”) is an open-label, multicenter, Phase 1 study to evaluate the safety and tolerability of the bispecific in subjects with relapsed or refractory CD20+ NHL who have progressed on standard anticancer therapy or for whom no other approved conventional therapy exists.

The study is conducted in 2 parts: Part A, monotherapy dose escalation and Part B, monotherapy dose expansion. Parts A and B consist of 3 periods: screening, treatment, and follow-up.

The dose escalation portion of the study (Part A) evaluates the safety and tolerability of increasing dose levels of TPP-1360 in order to identify the MTD and/or the RP2D in subjects with R/R CD20+ NHL excluding subjects with CLL/SLL (chronic lymphocytic leukemia/small lymphocytic leukemia).

The monotherapy dose expansion portion of the study (Part B) further evaluates the safety, pharmacokinetics, and antitumor activity of the bispecific at the recommended Phase 2 dose in selected cohorts of subjects with diffuse large B cell lymphoma (DLBCL) and follicular lymphoma (FL).

All treatments are administered until clinically significant disease progression, unacceptable toxicity, or subject/investigator decision to withdraw.

Study Population: Subjects (male or female) ≥18 years of age, with CD20+ NHL who have progressed on (or not been able to tolerate due to medical comorbidities or unacceptable toxicity) standard anticancer therapy, or for whom no other approved conventional therapy exists, are enrolled in the study. Approximately 35 to 40 subjects with mandatory, paired biopsies are enrolled in Part A dose escalation. Approximately 60 subjects (30 per cohort) are enrolled in Part B dose expansion.

Study Treatments: The bispecific antibody is provided for IV administration. The bispecific is an immunoglobulin G1 (IgG1) bispecific antibody co-targeting CD47 and CD20, and is designed to bind CD20 with high affinity and CD47 with detuned affinity.

Inclusion criteria: Subjects must satisfy the following criteria to receive bispecific antibody treatment as part of the clinical study:

Subjects must have the following laboratory values:
a. Absolute neutrophil count (ANC) ≥1.0×10⁹/L without growth factor support for 7 days (14 days if on pegfilgrastim).
b. Hemoglobin (Hgb) ≥8 g/dL without transfusion for 14 days.
c. Platelets (plt) ≥75×10⁹/L without transfusion for 7 days.
d. Aspartate aminotransferase (AST/SGOT) and alanine aminotransferase (ALT/SGPT)≤2.5×Upper Limit of Normal (ULN) or ≤5.0×ULN if tumor is present in the liver.
e. Serum bilirubin ≤1.5×ULN.
f. Estimated serum creatinine clearance of ≥45 mL/min using the Cockcroft-Gault equation or measured creatinine clearance using 24-hour urine collection.
g. International normalized ratio (INR) <1.5×ULN and partial thromboplastin time (PTT) <1.5×ULN

Exclusion criteria: A subject must satisfy one or more of the following criteria to receive bispecific antibody treatment as part of a clinical study:
a. does not have Burkitt's or lymphoblastic lymphoma;
b. does not have chronic lymphocytic leukemia (CLL) or small lymphocytic lymphoma (SLL) including Richter's transformation;
c. does not have cancer with symptomatic central nervous system (CNS) involvement;
d. is not on chronic systemic immunosuppressive therapy or corticosteroids exceeding a total dose of 140 mg within 14 days of said administration;
e. does not have clinically significant graft-versus-host disease (GVHD);
f. has no history of class III or IV congestive heart failure (CHF) or severe non ischemic cardiomyopathy, unstable angina, myocardial infarction, or ventricular arrhythmia within the previous 6 months;
g. does not have inadequate cardiac function, defined as left ventricular ejection fraction (LVEF) <45% as assessed by echocardiogram (ECHO) or multiple uptake gated acquisition (MUGA) scan performed within 30 days of said administration;
h. has not received prior investigational therapy directed at CD47 or SIRPα;
i. has not had treatment with CAR-T therapy ≤4 weeks prior to said administration;
j. has not had prior systemic cancer-directed treatments or investigational modalities ≤5 half-lives or 4 weeks prior to said administration, whichever is shorter;
k. has not had major surgery ≤2 weeks prior to starting TPP-1360, and wherein said subject has recovered from any clinically significant effects of any recent surgery;
l. has not had autologous stem cell transplant ≤3 months prior to said administration;
m. has not had allogeneic stem cell transplant with either standard or reduced intensity conditioning ≤6 months prior to said administration;
n. does not have diagnosed primary immune deficiency disease;
o. does not have diagnosed active human immunodeficiency virus (HIV) infection; except where said subject has well controlled HIV with CD4 T-cell (CD4+) counts ≥350 cells/uL without opportunistic infection within 12 months prior to said administration;
p. does not have active hepatitis B virus (HBV) or hepatitis C virus (HCV) infection;
q. is not on ongoing treatment with chronic, therapeutic dosing of an anti-coagulant;
r. has no history of autoimmune hemolytic anemia or autoimmune thrombocytopenia;
s. has no history of concurrent second cancers requiring active, ongoing systemic treatment; and/or
t. has not had a live virus vaccine within at least 4 weeks prior to said administration.

In some cases, criteria a-t must be satisfied.

TABLE 2
Sequences

| SEQ ID NO: | Description | Amino Acid Sequence |
|---|---|---|
| 1 | TPP-1361 | NIQMTQSPSSLSASVGDRVTITCQASQDIHRYLSWFQQDPGTVPQHLIYRESRF |
| | CD47 VL | VDGVPSRFSGSGSGTEFTLTISSLOPEDFATYYCLQYDEFPYTFGGGTKVEIK |
| 2 | TPP-1361 | QMQLVQSGAEVKKPGSSVKVSCKASGFNIKDYYLHWVRQAPGQGLEWMGW |
| | CD47 VH | IDPDQGDTYYAQKFQGRVTITRDRSTSTAYMELASLTAEDTAVYYCNAAYGESS |
| | | YPMDYWGQGTLVTVSS |
| 3 | TPP-1360 | NIQMTQSPSSLSASVGDRVTITCRASQDIHRYLSWFQQKPGKVPKHLIYRESRFV |
| | CD47 VL | DGVPSRFSGSGSGTEFTLTISSLOPEDFATYYCLQYDEFPYTFGGGTKVEIK |
| 4 | TPP-1360 | QMQLVQSGAEVKKPGSSVKVSCKASGFNIKDYYLHWVRQAPGKGLEWMGWI |
| | CD47 VH | DPDQGDTYYAQKFQGRVTITRDRSTSTAYMELRSLRAEDTAVYYCNAAYGESSY |
| | | PMDYWGQGTLVTVSS |
| 5 | TPP-1367 | NIQMTQSPSSLSASVGDRVTITCRASQDIHRYLSWFQQKPGKVPKHLIYRANRL |
| | CD47 VL | VSGVPSRFSGSGSGTEFTLTISSLQPEDFATYYCLQYDEFPYTFGGGTKVEIK |
| 6 | TPP-1367 | QMQLVQSGAEVKKPGSSVKVSCKASGFNIKDYYLHWVRQAPGKGLEWMGWI |
| | CD47 VH | DPDQGDTYYAQKFQGRVTITRDRSTSTAYMELRSLRAEDTAVYYCNAAYGESSY |
| | | PMDYWGQGTLVTVSS |
| 7 | anti-CD20 VL | QIVLSQSPAILSASPGEKVTMTCRASSSVSYIHWFQQKPGSSPKPWIYATSNLAS |
| | | GVPVRFSGSGSGTSYSLTISRVEAEDAATYYCQQWTSNPPTFGGGTKLEIK |
| 8 | anti-CD20 VH | QVQLQQPGAELVKPGASVKMSCKASGYTFTSYNMHWVKQTPGRGLEWIGAI |
| | | YPGNGDTSYNQKFKGKATLTADKSSSTAYMQLSSLTSEDSAVYYCARSTYYGGD |
| | | WYFNVWGAGTTVTVSA |
| 9 | 90002 VL | NIQMTQSPSAMSASVGDRVTITCKASQDIHRYLSWFQQKPGKVPKHLIYRANR |
| | | LVSGVPSRFSGSGSGTEFTLTISSLOPEDFATYYCLQYDEFPYTFGGGTKVEIK |
| 10 | 90002 VH | QMQLVQSGAEVKKTGSSVKVSCKASGFNIKDYYLHWVRQAPGQALEWMGW |
| | | IDPDQGDTEYAQKFQDRVTITRDRSMSTAYMELSSLRSEDTAMYYCNAAYGSS |
| | | SYPMDYWGQGTTVTVSS |
| 11 | 90002 | NIQMTQSPSAMSASVGDRVTITCKASQDIHRYLSWFQQKPGKVPKHLIYRANR |
| | WHOLE LC/ | LVSGVPSRFSGSGSGTEFTLTISSLQPEDFATYYCLQYDEFPYTFGGGTKVEIKRTV |
| | IgG1 | AAPSVFIFPPSDEQLKSGTASVVCLLNNFYPREAKVQWKVDNALQSGNSQESV |
| | | TEQDSKDSTYSLSSTLTLSKADYEKHKVYACEVTHQGLSSPVTKSFNRGEC |
| 12 | 90002 | QMQLVQSGAEVKKTGSSVKVSCKASGFNIKDYYLHWVRQAPGQALEWMGW |
| | WHOLE HC/ | IDPDQGDTEYAQKFQDRVTITRDRSMSTAYMELSSLRSEDTAMYYCNAAYGSS |
| | IgG1 | SYPMDYWGQGTTVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISRTPEVTCVVV |
| | | DVSHEDPEVKFNWYVDGVEVHNAKTKPREEQYNSTYRVVSVLTVLHQDWLN |
| | | GKEYKCKVSNKALPAPIEKTISKAKGQPREPQVYTLPPSREEMTKNQVSLTCLVK |
| | | GFYPSDIAVEWESNGQPENNYKTTPPVLDSDGSFFLYSKLTVDKSRWQQGNVF |
| | | SCSVMHEALHNHYTQKSLSLSPGK |
| 13 | LC-WHOLE | QIVLSQSPAILSASPGEKVTMTCRASSSVSYIHWFQQKPGSSPKPWIYATSNLAS |
| | RITUXIMAB | GVPVRFSGSGSGTSYSLTISRVEAEDAATYYCQQWTSNPPTFGGGTKLEIKRTVA |
| | | APSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| 14 | HC-WHOLE | QVQLQQPGAELVKPGASVKMSCKASGYTFTSYNMHWVKQTPGRGLEWIGAI |
| | RITUXIMAB | YPGNGDTSYNQKFKGKATLTADKSSSTAYMQLSSLTSEDSAVYYCARSTYYGGD |
| | | WYFNVWGAGTTVTVSAASTKGPSVFPLAPSSKSTSGGTAALGCLVKDYFPEPV |
| | | TVSWNSGALTSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISRTPEVTCVVV |
| | | DVSHEDPEVKFNWYVDGVEVHNAKTKPREEQYNSTYRVVSVLTVLHQDWLN |
| | | GKEYKCKVSNKALPAPIEKTISKAKGQPREPQVYTLPPSREEMTKNQVSLTCLVK |
| | | GFYPSDIAVEWESNGQPENNYKTTPPVLDSDGSFFLYSKLTVDKSRWQQGNVF |
| | | SCSVMHEALHNHYTQKSLSLSPGK |
| 15 | anti-CD20 | QIVLSQSPAILSASPGEKVTMTCRASSSVSYIHWFQQKPGSSPKPWIYATSNLAS |
| | WHOLE LC | GVPVRFSGSGSGTSYSLTISRVEAEDAATYYCQQWTSNPPTFGGGTKLEIKRTVA |
| | | APSVAIFPPSDERLKSGTASVVCVLNNFYPREAKVQWKVDNALQSGNSQESVT |
| | | EQDSKDSTYSLSSRLTLSKADYEKHKVYACEVTHQGLSSPVTKSFNRGEC |
| 16 | anti-CD20 | QVQLQQPGAELVKPGASVKMSCKASGYTFTSYNMHWVKQTPGRGLEWIGAI |
| | WHOLE HC | YPGNGDTSYNQKFKGKATLTADKSSSTAYMQLSSLTSEDSAVYYCARSTYYGGD |
| | | WYFNVWGAGTTVTVSAASTKGPSVFPLAPSSKSTSGGTAWLGCEVTDYFPEPV |
| | | TVSWNSGALTSGVHTFPAVLESSGLYSLSSVVTVPSSSLGTQTYICNVNHKPSNT |
| | | KVDKKVEPKSCDKTHTCPPCPAPELLGGPSVFLFPPKPKDTLMISRTPEVTCVVV |
| | | DVSHEDPEVKFNWYVDGVEVHNAKTKPREEQYNSTYRVVSVLTVLHQDWLN |
| | | GKEYKCKVSNKALPAPIEKTISKAKGQPREPQVYVYPPSREEMTKNQVSLTCLVK |
| | | GFYPSDIAVEWESNGQPENNYKTTPPVLDSDGSFALVSKLTVDKSRWQQGNVF |
| | | SCSVMHEALHNHYTQKSLSLSPGK |
| 17 | TPP-1361 | NIQMTQSPSSLSASVGDRVTITCQASQDIHRYLSWFQQDPGTVPQHLIYRESRF |
| | CD47 WHOLE | VDGVPSRFSGSGSGTEFTLTISSLQPEDFATYYCLQYDEFPYTFGGGTKVEIKRTV |
| | LC | AAPSVFIFPPSDEELKSGTASVVCWLNNFYPREAKVQWKVDNALQSGNSEESV |
| | | TEQDSKDSTYSLSSTLELSKADYEKHKVYACEVTHQGLSSPVTKSFNRGEC |
| 18 | TPP-1361 | QMQLVQSGAEVKKPGSSVKVSCKASGFNIKDYYLHWVRQAPGQGLEWMGW |
| | CD47 WHOLE | IDPDQGDTYYAQKFQGRVTITRDRSTSTAYMELASLTAEDTAVYYCNAAYGESS |
| | HC | YPMDYWGQGTLVTVSSASTKGPSVFPLAPSSKSTSGGTAALGCLVKDYFPEPVT |
| | | VSWNSGALTSGVHTFPAVLKSSGLYSLSSVVTVPSSSLGTQTYICNVNHKPSNTK |
| | | VDKKVEPKSCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQDWLNGK |
| | | EYKCKVSNKALPAPIEKTISKAKGQPREPQVYVLPPSREEMTKNQVSLLCLVKGF |
| | | YPSDIAVEWESNGQPENNYLTWPPVLDSDGSFFLYSKLTVDKSRWQQGNVFS |
| | | CSVMHEALHNHYTQKSLSLSPGK |
| 19 | TPP-1360 | NIQMTQSPSSLSASVGDRVTITCRASQDIHRYLSWFQQKPGKVPKHLIYRESRFV |
| | CD47 WHOLE | DGVPSRFSGSGSGTEFTLTISSLOPEDFATYYCLQYDEFPYTFGGGTKVEIKRTVA |
| | LC | APSVFIFPPSDEELKSGTASVVCWLNNFYPREAKVQWKVDNALQSGNSEESVT |
| | | EQDSKDSTYSLSSTLELSKADYEKHKVYACEVTHQGLSSPVTKSFNRGEC |
| 20 | TPP-1360 | QMQLVQSGAEVKKPGSSVKVSCKASGFNIKDYYLHWVRQAPGKGLEWMGWI |
| | CD47 WHOLE | DPDQGDTYYAQKFQGRVTITRDRSTSTAYMELRSLRAEDTAVYYCNAAYGESSY |
| | HC | PMDYWGQGTLVTVSSASTKGPSVFPLAPSSKSTSGGTAALGCLVKDYFPEPVTV |
| | | SWNSGALTSGVHTFPAVLKSSGLYSLSSVVTVPSSSLGTQTYICNVNHKPSNTKV |
| | | DKKVEPKSCDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWLNGKE |
| | | YKCKVSNKALPAPIEKTISKAKGQPREPQVYVLPPSREEMTKNQVSLLCLVKGFY |
| | | PSDIAVEWESNGQPENNYLTWPPVLDSDGSFFLYSKLTVDKSRWQQGNVFSCS |
| | | VMHEALHNHYTQKSLSLSPGK |
| 21 | TPP-1367 | NIQMTQSPSSLSASVGDRVTITCRASQDIHRYLSWFQQKPGKVPKHLIYRANRL |
| | CD47 WHOLE | VSGVPSRFSGSGSGTEFTLTISSLQPEDFATYYCLQYDEFPYTFGGGTKVEIKRTV |
| | LC | AAPSVFIFPPSDEELKSGTASVVCWLNNFYPREAKVQWKVDNALQSGNSEESV |
| | | TEQDSKDSTYSLSSTLELSKADYEKHKVYACEVTHQGLSSPVTKSFNRGEC |
| 22 | TPP-1367 | QMQLVQSGAEVKKPGSSVKVSCKASGFNIKDYYLHWVRQAPGKGLEWMGWI |
| | CD47 WHOLE | DPDQGDTYYAQKFQGRVTITRDRSTSTAYMELRSLRAEDTAVYYCNAAYGESSY |
| | HC | PMDYWGQGTLVTVSSASTKGPSVFPLAPSSKSTSGGTAALGCLVKDYFPEPVTV |
| | | SWNSGALTSGVHTFPAVLKSSGLYSLSSVVTVPSSSLGTQTYICNVNHKPSNTKV |
| | | DKKVEPKSCDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWLNGKE |
| | | YKCKVSNKALPAPIEKTISKAKGQPREPQVYVLPPSREEMTKNQVSLLCLVKGFY |
| | | PSDIAVEWESNGQPENNYLTWPPVLDSDGSFFLYSKLTVDKSRWQQGNVFSCS |
| | | VMHEALHNHYTQKSLSLSPGK |
| 23 | anti-CD47 | RTVAAPSVFIFPPSDEELKSGTASVVCWLNNFYPREAKVQWKVDNALQSGNSE |
| | IgG1 LC | ESVTEQDSKDSTYSLSSTLELSKADYEKHKVYACEVTHQGLSSPVTKSFNRGEC |
| | Constant | |
| | Region | |
| 24 | anti-CD47 | ELKSGTASVVCWLNNFYPREAKVQWKVDNALQSGNSEESVTEQDSKDSTYSLS |
| | IgG1 LC | STLE |
| | Constant | |
| | Region- | |
| | underlined | |
| | portion of | |
| | SEQ ID NO: 23 | |
| 25 | anti-CD47 | ASTKGPSVFPLAPSSKSTSGGTAALGCLVKDYFPEPVTVSWNSGALTSGVHTFP |
| | Constant | AVLKSSGLYSLSSVVTVPSSSLGTQTYICNVNHKPSNTKVDKKVEPKSCDKTHTC |
| | IgG1 HC | PPCPAPELLGGPSVFLFPPKPKDTLMISRTPEVTCVVVDVSHEDPEVKFNWYVD |
| | Region | GVEVHNAKTKPREEQYNSTYRVVSVLTVLHQDWLNGKEYKCKVSNKALPAPIE |
| | | KTISKAKGQPREPQVYVLPPSREEMTKNQVSLLCLVKGFYPSDIAVEWESNGQP |
| | | ENNYLTWPPVLDSDGSFFLYSKLTVDKSRWQQGNVFSCSVMHEALHNHYTQK |
| | | SLSLSPGK |
| 26 | anti-CD47 | KSSGLYSLSSVVTVPSSSLGTQTYICNVNHKPSNTKVDKKVEPKSCDKTHTCPPC |
| | IgG1 HC | PAPELLGGPSVFLFPPKPKDTLMISRTPEVTCVVVDVSHEDPEVKFNWYVDGVE |
| | Constant | VHNAKTKPREEQYNSTYRVVSVLTVLHQDWLNGKEYKCKVSNKALPAPIEKTIS |
| | Region- | KAKGQPREPQVYVLPPSREEMTKNQVSLLCLVKGFYPSDIAVEWESNGQPENN |
| | underlined | YLTW |
| | portion of | |
| | SEQ ID NO: 25 | |
| 27 | anti-CD20 | RTVAAPSVAIFPPSDERLKSGTASVVCVLNNFYPREAKVQWKVDNALQSGNSQ |
| | IgG1 LC | ESVTEQDSKDSTYSLSSRLTLSKADYEKHKVYACEVTHQGLSSPVTKSFNRGEC |
| | Constant | |
| | Region | |
| 28 | anti-CD20 | AIFPPSDERLKSGTASVVCVLNNFYPREAKVQWKVDNALQSGNSQESVTEQDS |
| | IgG1 LC | KDSTYSLSSR |
| | Constant | |
| | Region- | |
| | underlined | |
| | portion of | |
| | SEQ ID NO: 27 | |
| 29 | anti-CD20 | ASTKGPSVFPLAPSSKSTSGGTAWLGCEVTDYFPEPVTVSWNSGALTSGVHTFP |
| | IgG1 HC | AVLESSGLYSLSSVVTVPSSSLGTQTYICNVNHKPSNTKVDKKVEPKSCDKTHTC |
| | Constant | PPCPAPELLGGPSVFLFPPKPKDTLMISRTPEVTCVVVDVSHEDPEVKFNWYVD |
| | Region | GVEVHNAKTKPREEQYNSTYRVVSVLTVLHQDWLNGKEYKCKVSNKALPAPIE |
| | | KTISKAKGQPREPQVYVYPPSREEMTKNQVSLTCLVKGFYPSDIAVEWESNGQP |
| | | ENNYKTTPPVLDSDGSFALVSKLTVDKSRWQQGNVFSCSVMHEALHNHYTQK |
| | | SLSLSPGK |
| 30 | anti-CD20 | WLGCEVTDYFPEPVTVSWNSGALTSGVHTFPAVLESSGLYSLSSVVTVPSSSLGT |
| | IgG1 HC | QTYICNVNHKPSNTKVDKKVEPKSCDKTHTCPPCPAPELLGGPSVFLFPPKPKDT |
| | Constant | LMISRTPEVTCVVVDVSHEDPEVKFNWYVDGVEVHNAKTKPREEQYNSTYRVV |
| | Region | SVLTVLHQDWLNGKEYKCKVSNKALPAPIEKTISKAKGQPREPQVYVYPPSREE |
| | portion | MTKNQVSLTCLVKGFYPSDIAVEWESNGQPENNYKTTPPVLDSDGSFALV |
| 31 | 90002 | KASQDIHRYLS |
| | VL CDR1 | |
| 32 | 90002 | RANRLVS |
| | VL CDR2 | |
| 33 | 90002 | LQYDEFPYT |
| | VL CDR3 | |
| 34 | 90002 | DYYLH |
| | VH CDR1 | |
| 35 | 90002 | WIDPDQGDTEYAQKFQD |
| | VH CDR2 | |
| 36 | 90002 | AAYGSSSYPMDY |
| | VH CDR3 | |
| 37 | Rituximab | RASSSVSYIH |
| | CD20 VL | |
| | CDR1 | |
| 38 | Rituximab | ATSNLAS |
| | CD20 VL | |
| | CDR2 | |
| 39 | Rituximab | QQWTSNPPT |
| | CD20 VL | |
| | CDR3 | |
| 40 | Rituximab | SYNMH |
| | CD20 VH | |
| | CDR1 | |
| 41 | Rituximab | AIYPGNGDTSYNQKFKG |
| | CD20 VH | |
| | CDR2 | |
| 42 | Rituximab | STYYGGDWYFNV |
| | CD20 VH | |
| | CDR3 | |
| 43 | TPP-1361 | QASQDIHRYLS |
| | CD47 VL | |
| | CDR1 | |
| 44 | TPP-1361 | RESRFVD |
| | CD47 VL | |
| | CDR2 | |
| 45 | TPP-1361 | LQYDEFPYT |
| | CD47 VL | |
| | CDR3 | |
| 46 | TPP-1361 | DYYLH |
| | CD47 VH | |
| | CDR1 | |
| 47 | TPP-1361 | WIDPDQGDTYYAQKFQG |
| | CD47 VH | |
| | CDR2 | |
| 48 | TPP-1361 | AYGESSYPMDY |
| | CD47 VH | |
| | CDR3 | |
| 49 | TPP-1360 | RASQDIHRYLS |
| | CD47 VL | |
| | CDR1 | |
| 50 | TPP-1360 | RESRFVD |
| | CD47 VL | |
| | CDR2 | |
| 51 | TPP-1360 | LQYDEFPYT |
| | CD47 VL | |
| | CDR3 | |
| 52 | TPP-1360 | DYYLH |
| | CD47 VH | |
| | CDR1 | |
| 53 | TPP-1360 | WIDPDQGDTYYAQKFQG |
| | CD47 VH | |
| | CDR2 | |
| 54 | TPP-1360 | AYGESSYPMDY |
| | CD47 VH | |
| | CDR3 | |
| 55 | TPP-1367 | RASQDIHRYLS |
| | CD47 VL | |
| | CDR1 | |
| 56 | TPP-1367 | RANRLVS |
| | CD47 VL | |
| | CDR2 | |
| 57 | TPP-1367 | LQYDEFPYT |
| | CD47 VL | |
| | CDR3 | |
| 58 | TPP-1367 | DYYLH |
| | CD47 VH | |
| | CDR1 | |
| 59 | TPP-1367 | WIDPDQGDTYYAQKFQG |
| | CD47 VH | |
| | CDR2 | |
| 60 | TPP-1367 | AYGESSYPMDY |
| | CD47 VH | |
| | CDR3 | |
| 61 | TPP-1362 | RASQGISSWLA |
| | CD47 VL | |
| | CDR1 | |
| 62 | TPP-1362 | AASVLES |
| | CD47 VL | |
| | CDR2 | |
| 63 | TPP-1362 | QQANSFPYT |
| | CD47 VL | |
| | CDR3 | |
| 64 | TPP-1362 | NFVMS |
| | CD47 VH | |
| | CDR1 | |
| 65 | TPP-1362 | TISGSGGSTYYADSVKG |
| | CD47 VH | |
| | CDR2 | |
| 66 | TPP-1362 | HYILRYFD |
| | CD47 VH | |
| | CDR3 | |
| 67 | TPP-1362 | DIQMTQSPSSVSASVGDRVTITCRASQGISSWLAWYQQKPGKAPKLLIYAASVL |
| | CD47 VL | ESGVPSRFSGSGSGTDFTLTISSLOPEDFATYYCQQANSFPYTFGQGTKLEIK |
| 68 | TPP-1362 | EVQLLESGGGLVQPGGSLRLSCAASGFTFPNFVMSWVRQAPGKGLEWVSTISG |
| | CD47 VH | SGGSTYYADSVKGRFTISRDNSKNMLYLQMNSLRAEDTAVYYCAKHYILRYFD |
| | | WLAGTLVTVSS |
| 69 | TPP-1362 | DIQMTQSPSSVSASVGDRVTITCRASQGISSWLAWYQQKPGKAPKLLIYAASVL |
| | CD47 WHOLE | ESGVPSRFSGSGSGTDFTLTISSLOPEDFATYYCQQANSFPYTFGQGTKLEIKRTV |
| | LC | AAPSVFIFPPSDEELKSGTASVVCWLNNFYPREAKVQWKVDNALQSGNSEESV |
| | | TEQDSKDSTYSLSSTLELSKADYEKHKVYACEVTHQGLSSPVTKSFNRGEC |
| 70 | TPP-1362 | EVQLLESGGGLVQPGGSLRLSCAASGFTFPNFVMSWVRQAPGKGLEWVSTISG |
| | CD47 WHOLE | SGGSTYYADSVKGRFTISRDNSKNMLYLQMNSLRAEDTAVYYCAKHYILRYFD |
| | HC | WLAGTLVTVSSASTKGPSVFPLAPSSKSTSGGTAALGCLVKDYFPEPVTVSWNS |
| | | GALTSGVHTFPAVLKSSGLYSLSSVVTVPSSSLGTQTYICNVNHKPSNTKVDKKV |
| | | EPKSCDKTHTCPPCPAPELLGGPSVFLFPPKPKDTLMISRTPEVTCVVVDVSHED |
| | | PEVKFNWYVDGVEVHNAKTKPREEQYNSTYRVVSVLTVLHQDWLNGKEYKCK |
| | | VSNKALPAPIEKTISKAKGQPREPQVYVLPPSREEMTKNQVSLLCLVKGFYPSDIA |
| | | VEWESNGQPENNYLTWPPVLDSDGSFFLYSKLTVDKSRWQQGNVFSCSVMH |
| | | EALHNHYTQKSLSLSPGK |
| 71 | 408 437 VL | NIQMTQSPSSLSASVGDRVTITCRASQDIHRYLSWFQQKPGKVPKHLIYRANRL |
| | | VSGVPSRFSGSGSGTEFTLTISSLOPEDFATYYCLQYDEFPYTFGGGTKVEIK |
| 72 | 408 437 VH | QMQLVQSGAEVKKPGSSVKVSCKASGFNIKDYYLHWVRQAPGKGLEWMGWI |
| | | DPDQGDTEYAQKFQGRVTITRDRSTSTAYMELRSLRAEDTAVYYCNAAYGSSSY |
| | | PMDYWGQGTLVTVSS |

All publications and patents referred to herein are incorporated by reference. Various modifications and variations of the described subject matter will be apparent to those skilled in the art without departing from the scope and spirit of the methods disclosed herein. Although the methods have been described in connection with specific embodiments, it should be understood that the disclosure as claimed should not be unduly limited to these embodiments. Indeed, various modifications for carrying out the methods disclosed herein are readily available to those skilled in the art and are intended to be within the scope of the following claims.

1. A method for the treatment of a lymphoid malignant neoplasm in a subject in need thereof comprising administering an effective amount of a bispecific antibody which comprises:
i) a Fab portion that binds CD47 comprising a light chain variable region (VL) comprising a VL CDR1 comprising the amino acid sequence QASQDIHRYLS (SEQ ID NO:43) or RASQDIHRYLS (SEQ ID NO:49); a VL CDR2 comprising the amino acid sequence RESRFVD (SEQ ID NO:50) or RANRLVS (SEQ ID NO:56); and a VL CDR3 comprising the amino acid sequence LQYDEFPYT (SEQ ID NO:51); and a heavy chain variable region (VH) comprising a VH CDR1 comprising the amino acid sequence DYYLH (SEQ ID NO:52); a VH CDR2 comprising the amino acid sequence WIDPDQGDTYYAQKFQG (SEQ ID NO:53); and a VH CDR3 comprising the amino acid sequence AYGESSYPMDY (SEQ ID NO:54); and,
ii) a Fab portion that binds CD20 comprising a light chain variable region (VL) region comprising a VL CDR1 comprising the amino acid sequence RASSSVSYIH (SEQ ID NO:37), a VL CDR2 comprising the amino acid sequence ATSNLAS (SEQ ID NO:38), and a VL CDR3 comprising the amino acid sequence QQWTSNPPT (SEQ ID NO:39); and a heavy chain variable region (VH) region comprising a VH CDR1 comprising the amino acid sequence SYNMH (SEQ ID NO:40), a VH CDR2 comprising the amino acid sequence AIYPGNGDTSYNQKFKG (SEQ ID NO:41), and STYYGGDWYFNV (SEQ ID NO:42).

2. The method according to claim 1 wherein the Fab portion that binds CD47 comprises a light chain variable region (VL) comprising, or consisting of, the amino acid sequence of SEQ ID NO:1, SEQ ID NO:3, or SEQ ID NO:5; and a heavy chain variable region (VH) comprising, or consisting of, SEQ ID NO:2, SEQ ID NO:4, or SEQ ID NO:6.

3. The method according to claim 2 wherein the bispecific antibody comprises an anti-CD47 light chain comprising, or consisting of, the amino acid sequence of SEQ ID NO:17, SEQ ID NO:19, or SEQ ID NO:21; and, an anti-CD47 heavy chain comprising, or consisting of, the amino acid sequence of SEQ ID NO:18, SEQ ID NO:20, or SEQ ID NO:22.

4. The method according to claim 3, wherein the bispecific antibody comprises an anti-CD47 heavy chain comprising, or consisting of, the amino acid sequence of SEQ ID NO:18, SEQ ID NO:20, or SEQ ID NO:22, but wherein said anti-CD47 heavy chain lacks the C-terminal lysine.

5. The method according to claim 3 wherein the bispecific IgG1 antibody comprises an anti-CD20 light chain comprising the amino acid sequence of SEQ ID NO:15 and an anti-CD20 heavy chain comprising the amino acid sequence of SEQ ID NO:16.

6. The method of claim 5, wherein the bispecific antibody comprises an anti-CD20 heavy chain comprising, or consisting of, the amino acid sequence of SEQ ID NO:16, but wherein said anti-CD20 heavy chain lacks the C-terminal lysine.

7. The method according to claim 5 wherein the bispecific antibody comprises an anti-CD47 light chain comprising, or consisting of, the amino acid sequence of SEQ ID NO:19; and an anti-CD47 heavy chain comprising, or consisting of, the amino acid sequence of SEQ ID NO:20.

8. The method of claim 7, wherein the bispecific antibody comprises an anti-CD47 heavy chain comprising, or consisting of, the amino acid sequence of SEQ ID NO:20, but wherein said anti-CD47 heavy chain lacks the C-terminal lysine.

9. The method according to claim 1 wherein the lymphoid malignant neoplasm is Non-Hodgkin's Lymphoma (NHL), follicular lymphoma (FL), diffuse large B-cell lymphoma (DLBCL), marginal zone lymphoma (MZL), mantle cell lymphoma (MCL), or primary mediastinal B-cell lymphoma.

10. The method according to claim 9 wherein the subject has relapsed or refractory lymphoid malignant neoplasm.

11. The method of claim 10, wherein said lymphoid malignant neoplasm has progressed on standard anticancer therapy.

12. The method of claim 10, wherein or no other approved conventional therapy exists for said subject having said lymphoid malignant neoplasm.

13. The method according claim 10, wherein the lymphoid malignant neoplasm is NHL.

14. The method according to claim 13 wherein the lymphoid malignant neoplasm is relapsed or refractory NHL.

15. The method of claim 9, wherein said lymphoid malignant neoplasm is follicular lymphoma.

16. The method of claim 9, wherein said lymphoid malignant neoplasm is diffuse large B-cell lymphoma.

17. The method of claim 9, wherein said lymphoid malignant neoplasm is marginal zone lymphoma.

18. The method of claim 9, wherein said lymphoid malignant neoplasm is mantle cell lymphoma.

19. The method of claim 9, wherein said lymphoid malignant neoplasm is primary mediastinal B-cell lymphoma.

20. The method of claim 13, wherein said subject has at least one nodal lesion >1.5 cm in its longest diameter, or at least one extranodal lesion >1.0 cm in its longer diameter, on cross sectional imaging by CT or MRI as defined by Lugano criteria.

21. The method of claim 1, wherein said subject has one or more of:
a. absolute neutrophil count (ANC) ≥1.0×10⁹/L without growth factor support for 7 days (14 days if on pegfilgrastim);
b. hemoglobin (Hgb) ≥8 g/dL without transfusion for 14 days;
c. platelets (plt) ≥75×10⁹/L without transfusion for 7 days;
d. aspartate aminotransferase (AST/SGOT) and alanine aminotransferase (ALT/SGPT) ≤2.5×Upper Limit of Normal (ULN) or ≤5.0×ULN if tumor is present in the liver;
e. serum bilirubin ≤1.5×ULN;
f. estimated serum creatinine clearance of ≥45 mL/min using the Cockcroft-Gault equation or measured creatinine clearance using 24-hour urine collection; and/or
g. international normalized ratio (INR) <1.5×ULN and partial thromboplastin time (PTT) <1.5×ULN.

22. The method of claim 1, wherein said subject:
a. does not have Burkitt's or lymphoblastic lymphoma;
b. does not have chronic lymphocytic leukemia (CLL) or small lymphocytic lymphoma (SLL) including Richter's transformation;
c. does not have cancer with symptomatic central nervous system (CNS) involvement;
d. is not on chronic systemic immunosuppressive therapy or corticosteroids exceeding a total dose of 140 mg within 14 days of said administration;
e. does not have clinically significant graft-versus-host disease (GVHD);
f. has no history of class III or IV congestive heart failure (CHF) or severe non ischemic cardiomyopathy, unstable angina, myocardial infarction, or ventricular arrhythmia within the previous 6 months;
g. does not have inadequate cardiac function, defined as left ventricular ejection fraction (LVEF) <45% as assessed by echocardiogram (ECHO) or multiple uptake gated acquisition (MUGA) scan performed within 30 days of said administration;
h. has not received prior investigational therapy directed at CD47 or SIRPα;
i. has not had treatment with CAR-T therapy ≤4 weeks prior to said administration;
j. has not had prior systemic cancer-directed treatments or investigational modalities ≤5 half-lives or 4 weeks prior to said administration, whichever is shorter;
k. has not had major surgery ≤2 weeks prior to starting TPP-1360, and wherein said subject has recovered from any clinically significant effects of any recent surgery;
l. has not had autologous stem cell transplant ≤3 months prior to said administration;
m. has not had allogeneic stem cell transplant with either standard or reduced intensity conditioning ≤6 months prior to said administration;
n. does not have diagnosed primary immune deficiency disease;
o. does not have diagnosed active human immunodeficiency virus (HIV) infection; except where said subject has well controlled HIV with CD4+ T-cell (CD4+) counts ≥350 cells/uL without opportunistic infection within 12 months prior to said administration;
p. does not have active hepatitis B virus (HBV) or hepatitis C virus (HCV) infection;
q. is not on ongoing treatment with chronic, therapeutic dosing of an anti-coagulant;
r. has no history of autoimmune hemolytic anemia or autoimmune thrombocytopenia;
s. has no history of concurrent second cancers requiring active, ongoing systemic treatment; and/or
t. has not had a live virus vaccine within at least 4 weeks prior to said administration.
